(19) United States
(12) Patent Application Publication
(10) Pub. No.: US 2026/0174687 A1
(43) Pub. Date: Jun. 25, 2026

(54) PRODUCTS WITH SPHERICAL FILLER

(71) Applicants: ODEN Ross JAY, Winston-Salem, NC (US); NICOVENTURES TRADING LIMITED, London (GB)

(72) Inventors: Ross Jay Oden, Winston-Salem, NC (US); Michael S. Daniel, Winston-Salem, NC (US); Cortney R. Jackson, Winston-Salem, NC (US); Joshua Lee Watts, Winston-Salem, NC (US); Shahin Roohinejad, Winston-Salem, NC (US); Michael Zawadzki, Clemmons, NC (US); Stuart Martin, Southampton (GB); Fiona Aliu, Southampton (GB)

(21) Appl. No.: 19/125,871

(22) PCT Filed: Oct. 31, 2023
(86) PCT No.: PCT/IB2023/060981

§ 371 Date: Apr. 30, 2025

Related U.S. Application Data
(60) Provisional application No. 63/421,462, filed on Nov. 1, 2022.

(51) Int. Cl. A61K 9/00; A24B 13/00

(52) U.S. Cl. CPC A61K 9/009; A24B 13/00; A61K 9/0056

(57) ABSTRACT

The present disclosure provides pouched products that include an outer water-permeable pouch defining a cavity; and a composition adapted for oral use situated within the cavity, the composition including at least one water-soluble component, a substantially spherical filler component in an amount of about 60% or more by dry weight of the composition, and water in an amount of about 30% or less by weight of the composition, wherein the average particle size of the substantially spherical filler component is about 350 microns to about 650 microns.

### FIELD OF THE DISCLOSURE

The present disclosure relates to flavored products intended for human use. The products are configured for oral use and deliver substances such as flavors and/or active ingredients during use. Such products may include tobacco or a product derived from tobacco, or may be tobacco-free alternatives.

### BACKGROUND

There are many categories of products intended for oral use and enjoyment. For example, oral tobacco products containing nicotine, which is known to have both stimulant and anxiolytic properties, have been available for many years. Conventional formats for so-called “smokeless” tobacco products include moist snuff, snus, and chewing tobacco, which are typically formed almost entirely of particulate, granular, or shredded tobacco, and which are either portioned by the user or presented to the user in individual portions, such as in single-use pouches or sachets. See for example, the types of smokeless tobacco formulations, ingredients, and processing methodologies set forth in U.S. Pat. No. 6,668,839 to Williams; U.S. Pat. No. 6,834,654 to Williams; U.S. Pat. No. 6,953,040 to Atchley et al.; U.S. Pat. No. 7,032,601 to Atchley et al.; and 7,694,686 to Atchley et al.; U.S. Pat. No. 7,810,507 to Dube et al.; U.S. Pat. No. 7,819,124 to Strickland et al.; U.S. Pat. No. 7,861,728 to Holton, Jr. et al.; 7,901,512 to Quinter et al.; U.S. Pat. No. 8,627,828 to Strickland et al.; U.S. Pat. No. 11,246,334 to Atchley, each of which is incorporated herein by reference.

In addition, traditional tobacco materials and non-tobacco materials have been combined with other ingredients to form product formats distinct from traditional smokeless products, with example formats including pouched products, lozenges, pastilles, gels, and the like. See, for example, the types of products described in US Patent App. Pub. Nos. 2008/0196730 to Engstrom et al.; 2008/0305216 to Crawford et al.; 2009/0293889 to Kumar et al.; 2010/0291245 to Gao et al; 2011/0139164 to Mua et al.; 2012/0037175 to Cantrell et al.; 2012/0055494 to Hunt et al.; 2012/0138073 to Cantrell et al.; 2012/0138074 to Cantrell et al.; 2013/0074855 to Holton, Jr.; 2013/0074856 to Holton, Jr.; 2013/0152953 to Mua et al.; 2013/0274296 to Jackson et al.; 2015/0068545 to Moldoveanu et al.; 2015/0101627 to Marshall et al.; and 2015/0230515 to Lampe et al., each of which is incorporated herein by reference. There is continuing interest in the development of new types of oral products that deliver advantageous sensorial or biological activity. Such products typically contain flavorants and/or active ingredients such as nicotine, caffeine, botanicals, or cannabidiol. The format of such products can vary, and include pouched products containing a powdered or granular composition, lozenges, pastilles, liquids, gels, emulsions, meltable compositions, and the like. See, for example, the types of products described in US Patent App. Pub. Nos. 2022/0160675 to Gerardi et al.; 2022/0071984 to Poole et al.; 2021/0378948 to Gerardi et al.; 2021/0330590 to Hutchens et al.; 2021/0186081 to Gerardi et al.; 2021/0177754 to Keller et al; 2021/0177043 to Gerardi et al.; 2021/0177038 to Gerardi et al.; 2021/0169867 to Holton, Jr. et al.; 2021/0169792 to Holton, Jr. et al.; 2021/0169132 to Holton, Jr. et al.; 2021/0169121 to St. Charles, and 2021/0169122 to St. Charles, each of which is incorporated herein by reference

### BRIEF SUMMARY

The present disclosure relates to products incorporating one or more spherical filler components. Some such products into which these components are incorporated according to the present disclosure are pouched products including an outer water-permeable pouch defining a cavity containing a composition comprising a water-soluble component capable of being released through the water-permeable pouch.

In certain embodiments, the composition within the cavity of the pouch can contain a tobacco-derived product, such as a particulate tobacco material, nicotine, particulate non-tobacco material (e.g., microcrystalline cellulose) that has been treated to contain nicotine and/or flavoring agents, or fibrous plant material (e.g., beet pulp fiber) treated to contain a tobacco extract. In various embodiments, the composition within the cavity of the pouch is a smokeless tobacco product or nicotine replacement therapy product. In some embodiments, the composition within the cavity of the pouch can be a particulate material adapted for steeping or brewing (i.e., configured for liquid extraction), such as a tea or coffee material. Accordingly, in certain embodiments, the composition within the cavity of the pouch can comprise a particulate or fibrous plant material such as would be found in various teas or tea variants. In some embodiments, the composition within the cavity can comprise a flavor component such that flavor can be added to a liquid (e.g., water).

In some embodiments, pouched products are provided wherein the composition contained therein includes a substantially spherical filler component described herein above as the sole particulate filler component. In other embodiments, pouched compositions are provided which comprise one or more additional particulate filler components in addition to the substantially spherical filler component.

The invention includes, without limitation, the following embodiments.

Embodiment 1: A pouched product comprising: an outer water-permeable pouch defining a cavity; and a composition adapted for oral use situated within the cavity and comprising a water-soluble component; wherein the composition comprises a filler comprising spherical microcrystalline cellulose (“MCC”), and wherein the product has a water content of about 5% to about 50%.

Embodiment 2: The pouched product of Embodiment 1, wherein the composition comprises the spherical MCC in an amount of about 5% to about 95% by dry weight.

Embodiment 3: The pouched product of any of Embodiments 1-2, wherein the composition comprises the spherical MCC in an amount of about 25% to about 95% by dry weight or about 50% to about 95% by dry weight.

Embodiment 4: The pouched product of any of Embodiments 1-3, wherein the composition comprises the spherical MCC in an amount of about 60% to about 90% by dry weight.

Embodiment 5: The pouched product of any of Embodiments 1-4, wherein the composition further comprises at least one of a particulate tobacco material, an active ingredient, particulate non-tobacco material treated to contain an active ingredient and/or a flavoring agent, and fibrous plant material carrying a tobacco extract.

Embodiment 6: The pouched product of any of Embodiments 1-5, wherein the composition further comprises whitened tobacco material.

Embodiment 7: The pouched product of any of Embodiments 1-5, wherein the composition is substantially free of a tobacco material.

Embodiment 8: The pouched product of any of Embodiments 1-7, wherein the composition comprises one or more active ingredients selected from the group consisting of a nicotinic component, nutraceuticals, botanicals, stimulants, amino acids, vitamins, cannabinoids, cannabamimetics, terpenes, pharmaceutical agents, and combinations thereof.

Embodiment 9: The pouched product of any of Embodiments 1-8, wherein the composition comprises a nicotinic component selected from the group consisting of nicotine, a nicotine salt, or a resin complex of nicotine.

Embodiment 10: The pouched product of any of Embodiments 1-9, wherein the composition comprises a tobacco extract comprising nicotine.

Embodiment 11: The pouched product of any of Embodiments 1-10, wherein the composition comprises one or more components selected from the group consisting of one or more additional fillers, binders, pH adjusters, buffering agents, colorants, disintegration aids, antioxidants, humectants, and preservatives.

Embodiment 12: The pouched product of any of Embodiments 1-11, wherein the composition further comprises one or more of a sweetener, a salt, and a flavorant.

Embodiment 13: The pouched product of Embodiment 12, wherein the sweetener is selected from xylitol, sucralose, acesulfame-K, and combinations thereof.

Embodiment 14: The pouched product of any of Embodiments 1-13, wherein the composition comprises a humectant and the humectant comprises glycerin.

Embodiment 15: The pouched product of Embodiment 14, wherein the composition comprises about 2% to about 10% by weight of the glycerin.

Embodiment 16: The pouched product of any of Embodiments 1-15, further comprising one or more encapsulated additives.

Embodiment 17: The pouched product of any of Embodiments 1-16, having a water content of about 32% or less by weight.

Embodiment 18: The pouched product of any of Embodiments 1-17, having a water content of about 30% or less by weight.

Embodiment 19: The pouched product of any of Embodiments 1-18, having a water content of about 25% or less by weight.

Embodiment 20: The pouched product of any of Embodiments 1-19, having a water content of about 20% or less by weight.

Embodiment 21: The pouched product of any of Embodiments 1-20, having a water content of about 18% or less by weight.

Embodiment 22: The pouched product of any of Embodiments 1-21, having a water content of about 15% or less by weight.

Embodiment 23: The pouched product of any of Embodiments 1-22, wherein the spherical MCC comprises a coating layer comprising one or more components selected from the group consisting of a polyol, a functional cellulose, a filler, an active ingredient, a flavor, and combinations thereof.

Embodiment 24: A pouched product, comprising: an outer water-permeable pouch defining a cavity; and a composition adapted for oral use situated within the cavity, the composition comprising at least one water-soluble component, a substantially spherical filler component in an amount of about 60% or more by dry weight of the composition, and water in an amount of about 30% or less by weight of the composition, wherein the average particle size of the substantially spherical filler component is 350 microns to 650 microns.

Embodiment 25: The pouched product of Embodiment 24, wherein the average particle size of the substantially spherical filler component is 375 microns to 625 microns.

Embodiment 26: The pouched product of Embodiment 24 or 25, wherein water is present in an amount of about 5% to about 30% by weight of the composition.

Embodiment 27: The pouched product of any one of Embodiments 24-26, wherein water is present in an amount of about 15% to about 26% by weight of the composition.

Embodiment 28: The pouched product of any one of Embodiments 24-27, further comprising at least one humectant in an amount of about 0.1% by weight or higher by weight of the composition.

Embodiment 29: The pouched product of any one of Embodiments 24-28, wherein the at least one humectant is present in an amount of about 0.5% by weight to about 15% by weight of the composition.

Embodiment 30: The pouched product of any one of Embodiments 24-29, wherein the at least one humectant is propylene glycol, glycerin, or a combination thereof, or wherein the at least one humectant is glycerin in the absence of propylene glycol.

Embodiment 31: The pouched product of any one of Embodiments 24-30, further comprising at least one sugar alcohol.

Embodiment 32: The pouched product of any one of Embodiments 24-31, wherein the at least one sugar alcohol comprises xylitol.

Embodiment 33: The pouched product of any one of Embodiments 24-32, wherein xylitol is present in an amount of about 0.1 to about 5% by weight of the composition.

Embodiment 34: The pouched product of any one of Embodiments 24-33, wherein the composition further comprises a sweetener, a salt, and a flavorant.

Embodiment 35: The pouched product of any one of Embodiments 24-34, wherein the substantially spherical filler component comprises microcrystalline cellulose.

Embodiment 36: The pouched product of any one of Embodiments 24-35, wherein the composition is substantially free of a tobacco material.

Embodiment 37: The pouched product of any one of Embodiments 24-36, wherein the composition comprises one or more active ingredients selected from the group consisting of a nicotinic component, nutraceuticals, botanicals, stimulants, amino acids, vitamins, cannabinoids, cannabamimetics, terpenes, pharmaceutical agents, and combinations thereof.

Embodiment 38: The pouched product of any one of Embodiments 24-37, wherein the composition comprises a nicotinic component selected from the group consisting of nicotine, a nicotine salt, a resin complex of nicotine, and combinations thereof.

Embodiment 39: The pouched product of any one of Embodiments 24-38, wherein the pouched product exhibits a nicotine dissolution rate characterized by at least 80% by weight of the nicotine within the product calculated as the free base eluting from the product within 20 minutes as determined by high performance liquid chromatography (HPLC) analysis.

Embodiment 40: The pouched product of any one of Embodiments 24-39, wherein the composition further comprises one or more components selected from the group consisting of one or more additional fillers, binders, pH adjusters, buffering agents, colorants, disintegration aids, antioxidants, and preservatives. Embodiment 41: The pouched product of any one of Embodiments 24-40, wherein the spherical MCC comprises a coating layer comprising one or more components selected from the group consisting of a polyol, a functional cellulose, a filler, an active ingredient, a flavor, and combinations thereof.

Embodiment 42: The pouched product of any one of Embodiments 24-41, wherein the substantially spherical filler component comprises microcrystalline cellulose and has an average particle size of about 375 microns to about 625 microns, and the composition further comprises at least one humectant in an amount of about 0.5% by weight to about 15% by weight of the composition, xylitol in an amount of about 0.1 to about 5% by weight of the composition, and a nicotinic component in an amount of about 0.1% to about 10% by weight of the composition calculated as the free base.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

Having thus described aspects of the disclosure in the foregoing general terms, reference will now be made to the accompanying drawings, which are not necessarily drawn to scale. The drawings are examples only, and should not be construed as limiting the disclosure.

FIG. 1 is a front perspective view illustrating a pouched product according to an embodiment of the present disclosure;

FIGS. 2A and 2B are comparative scanning electron microscopy (SEM) images of non-limiting examples of conventional particulate filler material (A) and substantially spherical particulate filler material with 350 μm average particle size (B), respectively;

FIG. 3 illustrates the nicotine dissolution profile of two pouched product embodiments of the present disclosure as encompassed in Examples 1 and 2 in comparison to pouched products made using non-spherical filler materials;

FIG. 4 illustrates the nicotine dissolution profile of several pouched products of Example 4; and

FIG. 5 illustrates the nicotine dissolution profile of several pouched products of Example 7.

### DETAILED DESCRIPTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise.

The disclosure generally provides compositions and products configured for oral use. The term “configured for oral use” as used herein means that the product is provided in a form such that during use, saliva in the mouth of the user causes one or more of the components of the mixture (e.g., flavoring agents and/or active ingredients) to pass into the mouth of the user. In certain embodiments, the composition is adapted to deliver components to a user through mucous membranes in the user's mouth, the user's digestive system, or both, and, in some instances, said component is a nicotine component or an active ingredient (including, but not limited to, for example, nicotine, a stimulant, vitamin, amino acid, botanical, or a combination thereof) that can be absorbed through the mucous membranes in the mouth or absorbed through the digestive tract when the product is used.

In certain embodiments, the compositions and products provided herein are pouched products, e.g., in the form of a mixture of one or more components, disposed within a moisture-permeable container (e.g., a water-permeable pouch). Pouched products generally comprise, in addition to the pouch-based exterior, a composition/mixture within the pouch that typically comprises one or more active ingredients and/or one or more flavorants, and various other optional ingredients. According to the present disclosure, such composition/mixture further comprises a substantially spherical filler component. The composition/mixture can optionally include various other components; any remaining components of the composition within the pouches provided herein are not particularly limited. In some embodiments, the composition/mixture can comprise any filling composition, including those included within conventional pouched products, wherein at least a portion of any particulate filler component is replaced with the substantially spherical particulate filler described herein. Such compositions are generally mixtures of two or more components and as such, the compositions are, in some cases, referenced herein below as “mixtures.” Certain components that can advantageously be included in the mixtures within certain embodiments of the pouches provided herein are outlined generally below; however, it is to be understood that the discussion is not intended to be limiting of the components that can be incorporated within the disclosed pouches.

Such mixtures in the water-permeable pouch format are typically used by placing a pouch containing the mixture in the mouth of a human subject/user. Generally, the pouch is placed somewhere in the oral cavity of the user, for example under the lips, in the same way as moist snuff products are generally used. The pouch preferably is not chewed or swallowed. Exposure to saliva then causes some of the components of the mixture therein (e.g., flavoring agents and/or nicotine) to pass through e.g., the water-permeable pouch and provide the user with flavor and satisfaction, and the user is not required to spit out any portion of the mixture. After about 10 minutes to about 60 minutes, typically about 15 minutes to about 45 minutes, of use/enjoyment, substantial amounts of the mixture have been ingested by the human subject, and the pouch may be removed from the mouth of the consumer for disposal. Preferred pouch materials for products described herein may be designed and manufactured such that under conditions of normal use, a significant amount of the contents of the formulation within the pouch permeate through the pouch material prior to the time that the pouch undergoes loss of its physical integrity.

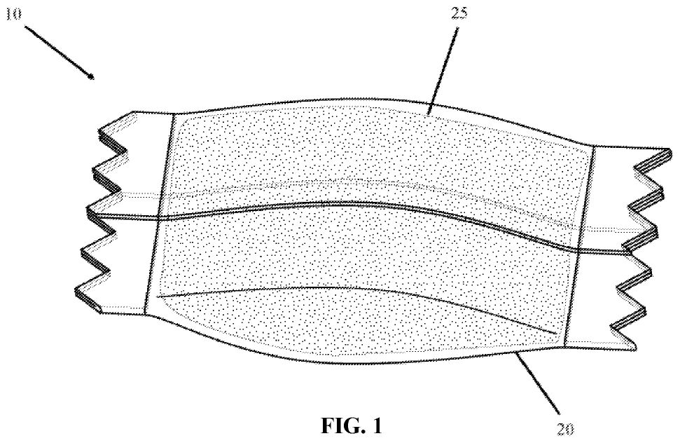

For example, as illustrated in FIG. 1, an example pouched product 10 can comprise an outer water-permeable container 20 in the form of a pouch which contains a particulate mixture 25 adapted for oral use. The orientation, size, and type of outer water-permeable pouch and the type and nature of the composition adapted for oral use that are illustrated herein are not construed as limiting thereof. Various, non-limiting components of certain particulate mixtures 25 according to the present disclosure are described in further detail herein below.

The material within the pouches as described herein typically includes at least one particulate filler component. Such particulate filler components may fulfill multiple functions, such as enhancing certain organoleptic properties such as texture and mouthfeel, enhancing cohesiveness or compressibility of the product, and the like.

According to the present disclosure, compositions provided herein (e.g. particulate mixture 25 of FIG. 1) comprise a substantially spherical particulate filler component (also referred to herein as a “substantially spherical filler component). By “substantially spherical” is meant at least a portion of the particulate filler component is in the shape of a sphere and/or is “sphere-like’ in shape. As such, “substantially spherical” encompasses slightly elongated (e.g., oval) shapes, slightly flattened shapes, and the like. Substantially spherical particulate filler components are intended to be distinguished from conventional particulate filler components (e.g., commercially available “fillers” or “particulate fillers” that are not explicitly designed as “spherical”). Such conventional particulate filler components typically substantially comprise particles with rather irregular shapes. In some cases, at least some (e.g., including a majority) of such conventional particulate filler components comprise one or more edges (e.g., jagged edges) that are typically not observable on substantially spherical particulate filler components as employed in the context of the present disclosure. Further, the uniformity of the particle shapes and sizes of a substantially spherical filler component is generally much greater than that of a conventional particulate filler component.

In certain embodiments of the present disclosure, substantially spherical fillers are defined by their sphericity, which is a measure of how closely an object resembles a perfect sphere. Sphericity (Ψ) can be measuring using the equation below, wherein Vₚ is the volume of the object and Aₚ is the surface area of the object.

Ψ=(π^{1/3}(6Vₚ)^{2/3})/Aₚ

The sphericity of a sphere is unity by definition and any shape that is not a perfect sphere will have a sphericity less than 1. In certain embodiments, the sphericity of the substantially spherical fillers of the present disclosure will be about 0.7 or higher, such as about 0.8 or higher or about 0.9 or higher (e.g., about 0.7 to 1 or about 0.75 to 1 or about 0.8 to 1 or about 0.85 to 1, or about 0.9 to 1).

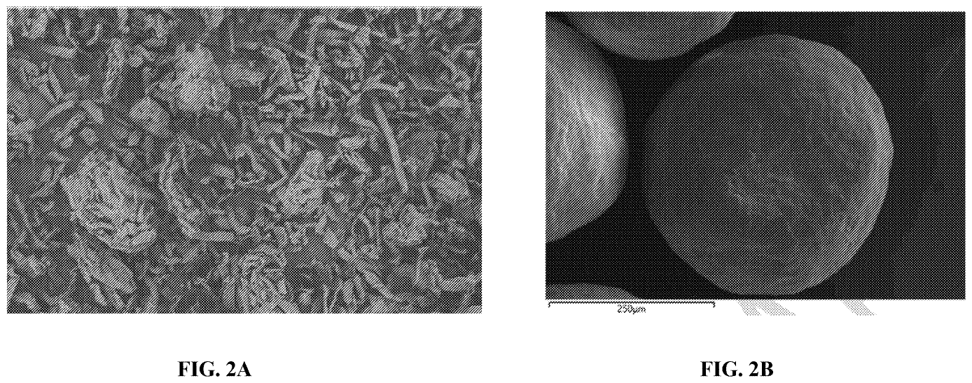

A non-limiting visual comparison between an example of a conventional microcrystalline cellulose (“MCC”) particulate filler component and an example of a substantially spherical MCC particulate filler component is provided as FIGS. 2A and 2B, respectively. As shown, the substantially spherical particulate filler is visually distinguishable from the conventional particulate filler.

The average diameter (mean) or D50 (median) particle size of the substantially spherical particulate filler particles provided herein can vary, and is not particularly limited. For example, in some embodiments, the spherical filler particles have an average diameter and/or a D50 value of about 100 μm to about 2000 μm, such as about 250 μm to about 750 μm. For example, in some embodiments, the average diameter is about 100 μm to about 500 μm, e.g., about 100 μm to about 400 μm, about 100 μm to about 300 μm, about 100 μm to about 200 μm, about 200 μm to about 500 μm, about 200 μm to about 400 μm, about 200 μm to about 300 μm, about 300 μm to about 500 μm, about 300 μm to about 400 μm, or about 400 μm to about 500 μm. In some embodiments, the average diameter is about 500 μm to about 1000 μm, e.g., about 500 μm to about 900 μm, about 500 μm to about 800 μm, about 500 μm to about 700 μm, about 500 μm to about 600 μm, about 600 μm to about 1000 μm, about 600 μm to about 900 μm, about 600 μm to about 800 μm, about 600 μm to about 700 μm, about 700 μm to about 1000 μm, about 700 μm to about 900 μm, about 700 μm to about 800 μm, about 800 μm to about 1000 μm, about 800 μm to about 900 μm, or about 900 μm to about 1000 μm.

The distribution of diameters around this average diameter (i.e., the particle size distribution) can also vary; in some embodiments, the distribution of diameters is close to the listed value (e.g., +/−about 25% of the stated value, +/−about 20% of the stated value, +/−about 15% of the stated value, +/−about 10% of the stated value, +/−about 5% of the stated value, or +/−about 1% of the stated value. The disclosure is not, however, limited to materials with such narrow distributions; in other embodiments, the diameter of the MCC spheres within a given material can vary within a wider range. Particle size distributions can be determined using a sieve analysis as explained hereinbelow.

In certain embodiments, the substantially spherical filler component has an average diameter and/or D50 value of 350 to 650 microns, such as 375 to 625 microns, or 400 to 600 microns, or 425 to 575 microns, or 425 to about 550 microns. For example, the substantially spherical filler component can have an average diameter and/or D50 value that is less than 700 microns or less than 675 microns or less than 650 microns, or an average diameter and/or D50 value that is greater than 300 microns, or greater than 325 microns, or greater than 350 microns. As shown in Example 9 below, nicotine dissolution of a pouched product made using a substantially spherical filler material having this size range is faster than similar products made using a substantially spherical filler having a size outside this range.

In some embodiments, the substantially spherical filler component comprises microcrystalline cellulose (“MCC”). The MCC may be synthetic or semi-synthetic, or it may be obtained entirely from natural celluloses. By “substantially spherical MCC” is meant a material comprising, consisting essentially of, or consisting of MCC, wherein the material is a substantially spherical particulate filler component as referenced herein above.

In some embodiments, the substantially spherical filler component comprises solid (although porous) MCC spheres. In some embodiments, the substantially spherical filler component comprises hollow MCC spheres. In some embodiments, the center/core of such hollow MCC spheres may be unfilled; in other embodiments, the center/core of such hollow MCC spheres may be filled with one or more additional components (e.g., flavorants, fillers, active ingredients, etc.).

The diameter of the substantially spherical MCC particles can vary within the scope of the disclosure, e.g., according to the values disclosed herein with respect to substantially spherical particulate filler components generally. Various commercial suppliers provide MCC spheres in designated diameter sizes and such commercial materials can be used in certain embodiments according to the present disclosure. Examples of suitable MCC spheres include, but are not limited to, Vivapur® MCC spheres from JRS Pharma, available, e.g., with particle sizes of 100-200 μm (Vivapur® 100), 200-355 μm (Vivapur® 200), 355-500 μm (Vivapur® 350), 500-710 μm (Vivapur® 500), 710-1000 μm (Vivapur®700), and 1000-1400 μm (Vivapur® 1000). Further examples of suitable MCC spheres include, but are not limited to, Celphere™ MCC spheres from Asahi Kasei Corporation, available, e.g., with particle sizes of 75-212 μm (Celphere™ SCP-100), 106-212 μm (Celphere™ CP-102), 150-300 μm (Celphere™ CP-203), 300-500 μm (Celphere™ CP-305), and 500-710 μm (Celphere™ CP-507). It is noted that this application focuses on the use of substantially spherical MCC; however, this disclosure is not meant to be limiting. Other types of materials that can serve the function of a filler as provided herein and which can be provided in substantially spherical form can be used in place of (or in addition) to the exemplified substantially spherical MCC filler without deviating from the scope of the disclosure.

The substantially spherical filler component is, in some embodiments, used in its pure form, i.e., the “spheres” consist essentially of or consist of the referenced filler material, e.g., MCC. As such, the “spheres” typically do not comprise any coating thereon, i.e., in some embodiments, the spheres are uncoated. It is noted that one or more of the other components of the mixture in which the substantially spherical filler component may, in some embodiments, be at least partially absorbed in and/or on at least a portion of the substantially spherical filler component, and such materials comprising “spheres” with a material (e.g., flavorant or active ingredient) absorbed/adsorbed in or on the spheres are to be included within the scope of this disclosure.

In some embodiments, the substantially spherical MCC particles are partially or wholly coated/encapsulated with an outer shell of one or more coatings (to give a core/shell-type structure), wherein the shell/coating include polyols or functional cellulose or other fillers (e.g., starches) as well as flavorants and active ingredients. For example, in a particular embodiment, the substantially spherical MCC particles are coated with a combination layer of nicotine and mannitol or a layer of nicotine and starch such as pea starch. In another particular embodiment, the substantially spherical MCC particles are coated with nicotine and HPMC. In one particular embodiment, the substantially spherical MCC particles are coated with a combination of flavorant and mannitol. In another particular embodiment, the substantially spherical MCC particles are coated with a combination of flavorant and HPMC. Also, certain embodiments can include substantially spherical MCC particles coated with a combination of nicotine, flavor, and mannitol. Other embodiments may include substantially spherical MCC particles coated with a combination of nicotine, flavor, and HPMC. Certain such coated and/or layered MCC spheres may have an average particle size and/or D50 of from about 350 to about 500 microns. The amount of coating can vary, with an example range of coating weight being about 10 to about 20% by weight, based on the total weight of the coated sphere. The method for applying a coating to the substantially spherical filler can vary. Example coating techniques utilize equipment such as pan coaters, fluidized bed coaters, and the like.

Advantageously, the use of a particulate filler component in substantially spherical form can endow the composition and product into which it is incorporated with unique physical properties. For example, in some embodiments, the replacement of at least a portion of conventional particulate filler component within a pouched composition with substantially spherical particulate filler component can allow for alternative moisture levels within the pouched composition as compared with compositions comprising all conventional particulate filler. For example, in some embodiments, use of the disclosed substantially spherical filler component can provide for a drier pouch than typically feasible using conventional filler components (e.g., all other components and amounts thereof being roughly equivalent). For example, in some embodiments, the disclosed composition, comprising a substantially spherical particulate filler component, can provide a pouched product having a moisture content/oven volatile content of about 32% by weight or less, such as about 30% or less, or about 28% or less, or about 27% or less, or about 26% or less, or about 25% or less, or about 24% or less, or about 23% or less, or about 22% or less, or about 21% or less, or about 20% or less, or about 19% or less, or about 18% or less, or about 17% or less, or about 16% or less, or about 15% or less. In some embodiments, the moisture content of the disclosed compositions can be about 5% by weight to about 32%, e.g., about 5% to about 25%, about 5% to about 20%, about 5% to about 15%, about 10% to about 25%, about 10% to about 20%, or about 10% to about 15%.

In other embodiments, the disclosed composition, comprising a substantially spherical particulate filler component, can provide a pouched product having a moisture content/oven volatile content of about 50% by weight or less, e.g., about 10% to about 50% or about 20% or about 50%. For example, the pouched product can have a moisture content/oven volatile content of about 5% by weight to about 50%, e.g., about 48% or below (e.g., including about 5% to about 48%). In some embodiments, the products have a moisture content that is above about 15% by weight, above about 20% by weight, above about 25% by weight, above about 30% by weight, or above about 40% by weight, based on the entirety of the pouched product. Certain examples of total moisture content according to certain embodiments include about 5% by weight to about 50%, about 5 to about 48%, about 10% to about 50%, about 10% to about 48%, about 15% to about 50%, about 15% to about 48%, about 25% to about 35%, about 25% to about 50%, about 25% to about 48%, about 30% to about 50%, about 30% to about 48%, about 30% to about 35%, about 40% to about 50%, about 40% to about 48%, about 45% to about 50%, or about 45% to about 48% by weight. Such total moisture content (also referred to as “oven volatiles”) includes, e.g., water in the composition within the pouched composition (which composition can have a moisture content, e.g., about 2% by weight to about 30% in various embodiments), additional water added to the product, e.g., sprayed onto the outside of the product after pouching, and humectants such as glycerin or propylene glycol in the composition. Thus, in some embodiments, the composition alone can also exhibit the moisture levels/oven volatile contents referenced herein above. Moisture/oven volatile content can be measured by drying a sample in an oven and precisely measuring the weight of the sample before and after drying so that the difference gives the amount of water or other oven volatiles evaporated (sometimes referred to as Loss on Drying or LOD).

In some embodiments, the provision of a drier pouch (e.g., about 32% by weight moisture or less, as referenced in the prior paragraph) due to the inclusion of particulate filler in the form of a substantially spherical particulate filler component can allow for different components to be suitably contained within the pouch as compared with inclusion of particulate filler in conventional form. For example, certain components that may desirably be included within a pouched composition may be more feasibly incorporated within a drier composition (e.g., where such components are water-soluble or water-reactive). For example, certain components encapsulated with a water-soluble exterior may more advantageously be included within a drier composition/product (e.g., a composition/product having a moisture content within the ranges referenced herein).

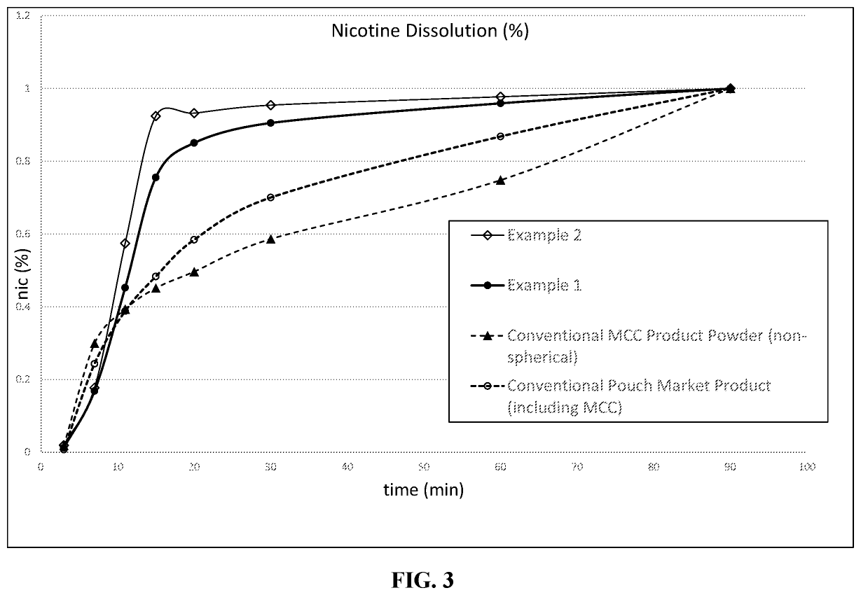

In some embodiments, the replacement of at least a portion of conventional particulate filler component within a pouched composition with substantially spherical particulate filler component can provide for modified release rates of active ingredients relative to compositions comprising all conventional particulate filler. At the same moisture level, a substantially spherical MCC component will generally release an active ingredient (e.g., nicotine) faster than a conventional particulate MCC component, as shown in FIGS. 3-5. Similarly, in some embodiments, at the same moisture level, a substantially spherical particulate filler component will release a flavorant faster than a conventional particulate filler component, as noted in the sensory data set forth in Example 6.

Some pouched product embodiments of the present disclosure can be characterized based on much higher nicotine dissolution rates as compared to pouched products made solely with conventional, non-spherical filler materials. For example, pouched products of the present disclosure can be characterized based on nicotine dissolution rate determined via high performance liquid chromatography (HPLC) analysis using Waters Acquity H-class system (or equivalent) with UV detection under the conditions set forth in the Experimental section herein. In certain embodiments, the pouched product exhibits a nicotine dissolution rate characterized by at least 80% by weight, such as at least 82% or at least 84% or at least 86%, or at least 88%, or at least 90% by weight, of the nicotine within the product eluted from the product within 20 minutes as determined by high performance liquid chromatography (HPLC) analysis.

As referenced above, the substantially spherical particulate filler component can be used as the sole particulate filler component within a pouched composition or can be used in combination with one or more other conventional particulate filler components (which can be the same base material or which can be a different base material than the substantially spherical particulate filler component. For example, in some embodiments, the substantially spherical filler component (e.g., substantially spherical MCC) is the only filler component within the pouched composition. In some embodiments, the substantially spherical filler component is substantially spherical MCC and the composition further comprises conventional (not substantially spherical) MCC, wherein the average particle size is roughly the same as the substantially spherical MCC or wherein the average particle size is different (i.e., larger or smaller) than the substantially spherical MCC. In some embodiments, the substantially spherical particulate filler component is MCC and the composition further comprises one or more conventional (not substantially spherical) particulate filler components (e.g., one or more starches or cellulose-based fillers other than MCC). Again, the one or more conventional filler components can have similar particle sizes and/or distributions as the substantially spherical filler components or can vary, e.g., in particle size (i.e., larger or smaller than the substantially spherical particulate filler component).

The particulate filler component(s), i.e., the substantially spherical filler component and, optionally, one or more additional particulate filler components, may fulfill multiple functions, such as enhancing certain organoleptic properties such as texture and mouthfeel, enhancing cohesiveness or compressibility of the product, and the like. Filler materials are typically insoluble in water.

### Additional Fillers

Generally, filler components are particulate materials and are cellulose-based. For example, suitable particulate filler components (both conventional and substantially spherical particulate filler components) are any non-tobacco plant material or derivative thereof, including cellulose materials derived from such sources. Examples of cellulosic non-tobacco plant material include cereal grains (e.g., maize, oat, barley, rye, buckwheat, and the like), sugar beet (e.g., FIBREX® brand filler available from International Fiber Corporation), bran fiber, and mixtures thereof. Non-limiting examples of derivatives of non-tobacco plant material include starches (e.g., from potato, wheat, rice, corn), natural cellulose, and modified cellulosic materials. Additional examples of potential particulate filler components include maltodextrin, dextrose, calcium carbonate, calcium phosphate, lactose, mannitol, xylitol, and sorbitol. Combinations of fillers can also be used.

“Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the mixture based on the ability of the starch material to impart a specific organoleptic property to composition. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties. Some starches have been developed by genetic modifications, and are considered to be “genetically modified” starches. Other starches are obtained and subsequently physically (e.g., heat, cool water swelling, etc.), chemically, or enzymatically modified. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, acetylation, hydroxypropylation, and/or partial hydrolysis. Enzymatic treatment includes subjecting native starches to enzyme isolates or concentrates, microbial enzymes, and/or enzymes native to plant materials, e.g., amylase present in corn kernels to modify corn starch. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, starch sodium octenyl succinate.

In some embodiments, the optional additional particulate filler component is a cellulose material or cellulose derivative and can, in some embodiments, comprise MCC (e.g., conventional/non-spherical MCC), although it is noted that, in some embodiments, employing combinations of substantially spherical MCC as provided herein with conventional/non-spherical MCC can, in some embodiments, result in segregation and/or dusting. The MCC may be synthetic or semi-synthetic, or it may be obtained entirely from natural celluloses. The MCC may be selected from the group consisting of AVICEL® grades PH-100, PH-102, PH-103, PH-105, PH-112, PH-113, PH-200, PH-300, PH-302, VIVACEL® grades 101, 102, 12, 20 and EMOCEL® grades 50M and 90M, and the like, and mixtures thereof.

In some embodiments, the particulate filler component further comprises a cellulose derivative or a combination of such derivatives. In some embodiments, the mixture comprises from about 1% to about 10% of the cellulose derivative by weight, based on the total weight of the mixture, with certain embodiments comprising about 1 to about 5% by weight of cellulose derivative. In certain embodiments, the cellulose derivative is a cellulose ether (including carboxyalkyl ethers), meaning a cellulose polymer with the hydrogen of one or more hydroxyl groups in the cellulose structure replaced with an alkyl, hydroxyalkyl, or aryl group. Non-limiting examples of such cellulose derivatives include methylcellulose, hydroxypropylcellulose (“HPC”), hydroxypropylmethylcellulose (“HPMC”), hydroxyethyl cellulose, and carboxymethylcellulose (“CMC”). In one embodiment, the cellulose derivative is one or more of methylcellulose, HPC, HPMC, hydroxyethyl cellulose, and CMC. In one embodiment, the cellulose derivative is HPC. In some embodiments, the mixture comprises from about 0% to about 5% HPC by weight, e.g., about 1% to about 3% HPC by weight, based on the total weight of the mixture.

In some further embodiments, the composition comprises, as a filler, a byproduct of a pulping process, such as citrus rinds. In some embodiments, the composition comprises, as a filler, wheat straw. Such fillers can be used in combination with any of the types of particulate fillers referenced herein above.

The overall amount of particulate filler component (including the substantially spherical particulate filler component and the optional additional particulate filler component(s)) can vary, but is typically up to about 90% of the material contained within the pouch by weight (i.e., the mixture), based on the total weight of the mixture. A typical range of particulate filler material within the mixture can be from about 10 to about 90 percent by total weight of the mixture, for example, from about 10, about 15, about 20, about 25, or about 30, to about 35, about 40, about 45, about 50, about 55, or about 60 weight percent (e.g., about 20 to about 50 weight percent, about 25 to about 45 weight percent, about 50 to about 90 weight percent or about 60 to about 90 weight percent). In certain embodiments, the amount of particulate filler material is at least about 10 percent by weight, such as at least about 20 percent, or at least about 25 percent, or at least about 30 percent, or at least about 35 percent, at least about 40 percent, at least about 50%, or at least about 60%, based on the total weight of the mixture.

In some embodiments, thus, the pouched composition comprises about 10% to about 95% by weight of the substantially spherical particulate filler component. The amount of substantially spherical particulate filler component incorporated within compositions and pouched products according to the disclosure can vary depending, e.g., upon the composition and physical parameters of the substantially spherical particulate filler, the type and size of the product into which it is incorporated, the other components of the product into which it is incorporated, and/or the desired characteristics to be achieved.

Where one or more additional particulate filler components is included within the pouched composition, the amount of substantially spherical particulate filler component can be scaled accordingly such that the overall amount of particulate filler falls within the ranges described above. The ratio of substantially spherical particulate filler component to conventional particulate filler component (where included) can vary across a wide range. The composition may, in some embodiments, comprise a greater amount by weight of the substantially spherical filler component, e.g., such that the particulate filler component of the composition in its entirety comprises about 50% to 100% by weight of the substantially spherical particulate (e.g., MCC) filler component (e.g., at least about 55%, at least about 60%, at least about 65%, at least about 70%, at least about 75%, at least about 80%, at least about 85%, at least about 90% by weight, at least about 95% by weight, or about 100% by weight), with any remainder comprising one or more conventional particulate filler components. The composition may, in some embodiments, comprise substantially similar amounts by weight of the substantially spherical particulate filler component and the conventional particulate filler component. The composition may, in some embodiments, comprise a lesser amount by weight of the substantially spherical filler component, e.g., such that the particulate filler component of the composition in its entirety comprises about 1% to about 50% by weight of the substantially spherical particulate filler component (e.g., about 10% to about 50% or about 25% to about 50% by weight), with the remainder comprising one or more conventional particulate filler components.

### Water

The water content of the mixture within the pouched product described herein, prior to use by a consumer of the product, may vary according to the desired properties. Typically, the mixture, as present within the product prior to insertion into the mouth of the user, is less than about 60 percent by weight of water, and generally is from about 1% to about 60% by weight of water, for example, from about 5% to about 55%, about 10% to about 50%, about 20% to about 45%, or about 25% to about 40% water by weight, including water amounts of at least about 5% by weight, at least about 10% by weight, at least about 15% by weight, and at least about 20% by weight.

In some embodiments, the mixture comprises a lower water content than some conventional mixtures for inclusion within a pouched product. For example, in some embodiments, the mixture comprises water in an amount of up to about 25% by weight or up to about 20% by weight, based on the total weight of the mixture. Example embodiments can include water in an amount of about 15% to about 25%, e.g., about 17% to about 20%.

### Flavoring Agent

As used herein, a “flavoring agent” or “flavorant” is any flavorful or aromatic substance capable of altering the sensory characteristics associated with the oral product. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy. Specific types of flavors include, but are not limited to, vanilla, coffee, chocolate/cocoa, cream, mint, spearmint, menthol, peppermint, wintergreen, eucalyptus, lavender, cardamon, nutmeg, cinnamon, clove, cascarilla, sandalwood, honey, jasmine, ginger, anise, sage, licorice, lemon, orange, apple, peach, lime, cherry, strawberry, trigeminal sensates, melatonin, terpenes, and any combinations thereof. See also, Leffingwell et al., Tobacco Flavoring for Smoking Products, R. J. Reynolds Tobacco Company (1972), which is incorporated herein by reference. Flavorings also may include components that are considered moistening, cooling or smoothening agents, such as eucalyptus. These flavors may be provided neat (i.e., alone) or in a composite, and may be employed as concentrates or flavor packages (e.g., spearmint and menthol, orange and cinnamon; lime, pineapple, and the like). Representative types of components also are set forth in U.S. Pat. No. 5,387,416 to White et al.; US Pat. App. Pub. No. 2005/0244521 to Strickland et al.; and PCT Application Pub. No. WO 05/041699 to Quinter et al., each of which is incorporated herein by reference. In some instances, the flavoring agent may be provided in a spray-dried form or a liquid form.

The flavoring agent generally comprises at least one volatile flavor component. As used herein, “volatile” refers to a chemical substance that forms a vapor readily at ambient temperatures (i.e., a chemical substance that has a high vapor pressure at a given temperature relative to a nonvolatile substance). Typically, a volatile flavor component has a molecular weight below about 400 Da, and often include at least one carbon-carbon double bond, carbon-oxygen double bond, or both. In one embodiment, the at least one volatile flavor component comprises one or more alcohols, aldehydes, aromatic hydrocarbons, ketones, esters, terpenes, terpenoids, or a combination thereof. Non-limiting examples of aldehydes include vanillin, ethyl vanillin, p-anisaldehyde, hexanal, furfural, isovaleraldehyde, cuminaldehyde, benzaldehyde, and citronellal. Non-limiting examples of ketones include 1-hydroxy-2-propanone and 2-hydroxy-3-methyl-2-cyclopentenone-1-one. Non-limiting examples of esters include allyl hexanoate, ethyl heptanoate, ethyl hexanoate, isoamyl acetate, and 3-methylbutyl acetate. Non-limiting examples of terpenes include sabinene, limonene, gamma-terpinene, beta-farnesene, nerolidol, thujone, myrcene, geraniol, nerol, citronellol, linalool, and eucalyptol. In one embodiment, the at least one volatile flavor component comprises one or more of ethyl vanillin, cinnamaldehyde, sabinene, limonene, gamma-terpinene, beta-farnesene, or citral. In one embodiment, the at least one volatile flavor component comprises ethyl vanillin.

The amount of flavoring agent, where present, utilized in the mixture/composition can vary, but is typically up to about 10 weight percent, and certain embodiments are characterized by a flavoring agent content of at least about 0.1 weight percent, such as about 0.1 to about 1 weight percent, 0.5 to about 10 weight percent, about 1 to about 6 weight percent, or about 2 to about 5 weight percent, based on the total weight of the mixture. The amount of flavoring agent present within the mixture may vary over a period of time (e.g., during a period of storage after preparation of the mixture). For example, certain volatile components present in the mixture may evaporate or undergo chemical transformations, leading to a reduction in the concentration of one or more volatile flavor components.

### Salts

In some embodiments, the mixture may further comprise a salt (e.g., alkali metal salts), typically employed in an amount sufficient to provide desired sensory attributes to the mixture. Non-limiting examples of suitable salts include sodium chloride, potassium chloride, ammonium chloride, flour salt, and the like. When present, a representative amount of salt is about 0.5 percent by weight or more, about 1.0 percent by weight or more, or at about 1.5 percent by weight or more, but will typically make up about 10 percent or less of the total weight of the mixture, or about 7.5 percent or less or about 5 percent or less (e.g., about 0.5 to about 5 percent by weight).

### Sweeteners

The mixture typically further comprises one or more sweeteners. The sweeteners can be any sweetener or combination of sweeteners, in natural or artificial form, or as a combination of natural and artificial sweeteners. Examples of natural sweeteners include isomaltulose, fructose, sucrose, glucose, maltose, mannose, galactose, lactose, stevia, honey, and the like. Examples of artificial sweeteners include sucralose, maltodextrin, saccharin, aspartame, acesulfame K, neotame and the like. In some embodiments, the sweetener comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). In certain embodiments, the mixture provided herein can include a sugar alcohol (e.g., xylitol or erythritol) in combination with a lesser amount of artificial sweetener (e.g., sucralose, aspartame, acesulfame K, or any combination thereof). When present, a representative amount of sweetener may make up from about 0.1 to about 20 percent or more of the of the mixture by weight, for example, from about 0.1 to about 1%, from about 1 to about 5%, from about 5 to about 10%, or from about 10 to about 20% of the mixture on a weight basis, based on the total weight of the mixture.

### Binding Agents

A binder (or combination of binders) may be employed in certain embodiments, in amounts sufficient to provide the desired physical attributes and physical integrity to the mixture. Binders also often function as thickening or gelling agents. Typical binders can be organic or inorganic, or a combination thereof. Representative binders include modified cellulose, povidone, sodium alginate, starch-based binders, pectin, carrageenan, pullulan, zein, and the like, and combinations thereof. In some embodiments, the binder comprises pectin or carrageenan or combinations thereof.

A binder may be employed in amounts sufficient to provide the desired physical attributes and physical integrity to the mixture. The amount of binder utilized in the mixture can vary, but is typically up to about 30 weight percent, and certain embodiments are characterized by a binder content of at least about 0.1% by weight, such as about 1 to about 30% by weight, or about 5 to about 10% by weight, based on the total weight of the mixture. In some embodiments, the composition/mixture provided herein is free of (i.e., does not contain) any binder component, e.g., gum.

In certain embodiments, the binder includes a gum, for example, a natural gum. As used herein, a natural gum refers to polysaccharide materials of natural origin that have binding properties, and which are also useful as a thickening or gelling agents. Representative natural gums derived from plants, which are typically water soluble to some degree, include xanthan gum, guar gum, gum arabic, ghatti gum, gum tragacanth, karaya gum, locust bean gum, gellan gum, and combinations thereof. When present, natural gum binder materials are typically present in an amount of up to about 5% by weight, for example, from about 0.1, about 0.2, about 0.3, about 0.4, about 0.5, about 0.6, about 0.7, about 0.8, about 0.9, or about 1%, to about 2, about 3, about 4, or about 5% by weight, based on the total weight of the mixture.

### Humectants

In certain embodiments, one or more humectants may be employed in the mixture. Examples of humectants include, but are not limited to, glycerin, propylene glycol, and the like. Where included, the humectant is typically provided in an amount sufficient to provide desired moisture attributes to the mixture. Further, in some instances, the humectant may impart desirable flow characteristics to the mixture for depositing in a mold. When present, a humectant will typically make up about 15% or less of the weight of the mixture or 10% or less of the weight of the mixture (e.g., from about 0.1% to about 15% by weight or about 0.5% to about 10% by weight). When present, a representative amount of humectant is about 0.1% to about 1% by weight, about 0.1% to about 0.5% by weight, or about 1% to about 5% by weight, based on the total weight of the mixture.

In certain embodiments, the composition is characterized by a relatively high amount of glycerin, which has been shown to enhance nicotine dissolution characteristics. For example, the amount of glycerin can be about 5% by weight or more, about 6% or more, about 7% or more, about 8% or more, about 9% or more, or about 10% or more (e.g., about 5% to about 15% or about 7.5% to about 14% or about 8% to about 12% by weight).

In certain embodiments the composition can be characterized by the relative absence of propylene glycol, which may impede nicotine dissolution in certain embodiments. For example, in certain embodiments, the composition comprises 2% by weight or less propylene glycol, such as 1.5% or less or 1.0% or less or 0.8% or less or 0.6% or less (e.g., about 0.1 to about 2% by weight or even 0.0% by weight, meaning no propylene glycol is intentionally added). In other embodiments, the weight ratio of glycerin to propylene glycol can be characterized as about 10:1 or higher, such as about 12:1 or higher or about 13:1 or higher or about 14:1 or higher or about 15:1 or higher or about 16:1 or higher or about 17:1 or higher (e.g., about 10:1 to about 25:1 or about 12:1 to about 18:1).

### Processing Aids

If necessary for downstream processing of the composition, such as granulation, mixing, or molding, a flow aid can also be added to the composition in order to enhance flowability of the composition. In some embodiments, the composition (e.g., melt and chew forms) may be surface treated with anti-stick agents, such as oils, silicones, and the like. Exemplary flow aids include microcrystalline cellulose, silica, polyethylene glycol, stearic acid, calcium stearate, magnesium stearate, zinc stearate, sodium stearyl fumarate, canauba wax, and combinations thereof. In some embodiments, the flow aid is sodium stearyl fumarate. When present, a representative amount of flow aid may make up at least about 0.5 percent or at least about 1 percent, of the total dry weight of the composition. Preferably, the amount of flow aid within the composition will not exceed about 5 percent, and frequently will not exceed about 3 percent, of the total dry weight of the mixture/composition.

### Buffering Agents

In certain embodiments, the mixture of the present disclosure can comprise pH adjusters or buffering agents. Examples of pH adjusters and buffering agents that can be used include, but are not limited to, metal hydroxides (e.g., alkali metal hydroxides such as sodium hydroxide and potassium hydroxide), and other alkali metal buffers such as metal carbonates (e.g., potassium carbonate or sodium carbonate), or metal bicarbonates such as sodium bicarbonate, and the like. Where present, the buffering agent is typically present in an amount less than about 5 percent based on the weight of the mixture, for example, from about 0.1% to about 1%, about 0.1% to about 0.5%, or 0.5% to about 5%, such as, e.g., from about 0.75% to about 4%, from about 0.75% to about 3%, or from about 1% to about 2% by weight, based on the total weight of the mixture. Non-limiting examples of suitable buffers include alkali metals acetates, glycinates, phosphates, glycerophosphates, citrates, carbonates, hydrogen carbonates, borates, or mixtures thereof.

### Oral Care Ingredient

Oral care ingredients provide the ability to inhibit tooth decay or loss, inhibit gum disease, relieve mouth pain, whiten teeth, or otherwise inhibit tooth staining, elicit salivary stimulation, inhibit breath malodor, freshen breath, or the like. For example, effective amounts of ingredients such as thyme oil, eucalyptus oil and zinc (e.g., such as the ingredients of formulations commercially available as ZYTEX® from Discus Dental) can be incorporated into the composition. Other examples of ingredients that can be incorporated in desired effective amounts within the present composition can include those that are incorporated within the types of oral care compositions set forth in Takahashi et al., Oral Microbiology and Immunology, 19 (1), 61-64 (2004); U.S. Pat. No. 6,083,527 to Thistle; and US Pat. Appl. Pub. Nos. 2006/0210488 to Jakubowski and 2006/02228308 to Cummins et al. Other exemplary ingredients of tobacco containing-formulation include those contained in formulations marketed as MALTISORB® by Roquette and DENTIZYME® by NatraRx. When present, a representative amount of oral care additive is at least about 1%, often at least about 3%, and frequently at least about 5% of the total dry weight of the composition. The amount of oral care additive within the composition will not typically exceed about 30%, often will not exceed about 25%, and frequently will not exceed about 20%, of the total dry weight of the mixture/composition.

### Colorants

A colorant may optionally be employed in amounts sufficient to provide the desired physical attributes to the mixture. Examples of colorants include various dyes and pigments, such as caramel coloring and titanium dioxide. The amount of colorant utilized in the mixture can vary, but when present is typically up to about 3 weight percent, such as from about 0.1%, about 0.5%, or about 1%, to about 3% by weight, based on the total weight of the mixture.

### Active Ingredient

The composition as disclosed herein includes one or more active ingredients. As used herein, an “active ingredient” refers to one or more substances belonging to any of the following categories: API (active pharmaceutical ingredient), food additives, natural medicaments, and naturally occurring substances that can have an effect on humans. Example active ingredients include any ingredient known to impact one or more biological functions within the body, such as ingredients that furnish pharmacological activity or other direct effect in the diagnosis, cure, mitigation, treatment, or prevention of disease, or which affect the structure or any function of the body of humans (e.g., provide a stimulating action on the central nervous system, have an energizing effect, an antipyretic or analgesic action, or an otherwise useful effect on the body). In some embodiments, the active ingredient may be of the type generally referred to as dietary supplements, nutraceuticals, “phytochemicals” or “functional foods.” These types of additives are sometimes defined in the art as encompassing substances typically available from naturally-occurring sources (e.g., botanical materials) that provide one or more advantageous biological effects (e.g., health promotion, disease prevention, or other medicinal properties), but are not classified or regulated as drugs.

Non-limiting examples of active ingredients include those falling in the categories of botanical ingredients, stimulants, amino acids, nicotine components, and/or pharmaceutical, nutraceutical, and medicinal ingredients (e.g., vitamins, such as A, B3, B6, B12, and C, and/or cannabinoids, such as tetrahydrocannabinol (THC) and cannabidiol (CBD)). Each of these categories is further described herein below. The particular choice of active ingredients will vary depending upon the desired flavor, texture, and desired characteristics of the particular product.

In certain embodiments, the active ingredient is selected from the group consisting of caffeine, taurine, GABA, theanine, vitamin C, lemon balm extract, ginseng, citicoline, sunflower lecithin, and combinations thereof. For example, the active ingredient can include a combination of caffeine, theanine, and optionally ginseng. In another embodiment, the active ingredient includes a combination of theanine, gamma-amino butyric acid (GABA), and lemon balm extract. In a further embodiment, the active ingredient includes theanine, theanine and tryptophan, or theanine and one or more B vitamins (e.g., vitamin B6 or B12). In a still further embodiment, the active ingredient includes a combination of caffeine, taurine, and vitamin C.

The particular percentages of active ingredients present will vary depending upon the desired characteristics of the particular product. Typically, an active ingredient or combination thereof is present in a total concentration of at least about 0.001% by weight of the composition, such as in a range from about 0.001% to about 20%. In some embodiments, the active ingredient or combination of active ingredients is present in a concentration from about 0.1% w/w to about 10% by weight, such as, e.g., from about 0.5% w/w to about 10%, from about 1% to about 10%, from about 1% to about 5% by weight, based on the total weight of the composition. In some embodiments, the active ingredient or combination of active ingredients is present in a concentration of from about 0.001%, about 0.01%, about 0.1%, or about 1%, up to about 20% by weight, such as, e.g., from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the composition. Further suitable ranges for specific active ingredients are provided herein below.

### Botanical

In some embodiments, the active ingredient comprises a botanical ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species). In some embodiments, the compositions as disclosed herein can be characterized as free of any tobacco material (e.g., any embodiment as disclosed herein may be completely or substantially free of any tobacco material). By “substantially free” is meant that no tobacco material has been intentionally added. For example, certain embodiments can be characterized as having less than 0.001% by weight of tobacco, or less than 0.0001%, or even 0% by weight of tobacco.

When present, a botanical is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the composition.

The botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein. Non-limiting examples of botanicals or botanical-derived materials include acai berry (Euterpe oleracea martius), acerola (Malpighia glabra), alfalfa, allspice, Angelica root, anise (e.g., star anise), annatto seed, apple (Malus domestica), apricot oil, ashwagandha, Bacopa monniera, baobab, basil (Ocimum basilicum), bay, bee balm, beet root, bergamot, blackberry (Morus nigra), black cohosh, black pepper, black tea, blueberries, boldo (Peumus boldus), borage, bugleweed, cacao, calamus root, camu (Myrcaria dubia), cannabis/hemp, caraway seed, cardamom, cassis, catnip, catuaba, cayenne pepper, Centella asiatica, chaga mushroom, Chai-hu, chamomile, cherry, chervil, chive, chlorophyll, chocolate, cilantro, cinnamon (Cinnamomum cassia), citron grass (Cymbopogon citratus), citrus, clary sage, cloves, coconut (Cocos nucifera), coffee, comfrey leaf and root, cordyceps, coriander seed, cranberry, cumin, curcumin, damiana, dandelion, Dorstenia arifolia, Dorstenia odorata, Echinacea, elderberry, elderflower, endro (Anethum graveolens), evening primrose, eucalyptus, fennel, feverfew, flax, Galphimia glauca, garlic, ginger (Zingiber officinale), gingko biloba, ginseng, goji berries, goldenseal, grape seed, grapefruit, grapefruit rosé (Citrus paradisi), graviola (Annona muricata), green tea, guarana, gutu kola, hawthorn, hazel, hemp, hibiscus flower (Hibiscus sabdariffa), honeybush, hops, jiaogulan, jambu (Spilanthes oleraceae), jasmine (Jasminum officinale), juniper berry (Juniperus communis), Kaempferia parviflora (Thai ginseng), kava, laurel, lavender, lemon (Citrus limon), lemon balm, lemongrass, licorice, lilac, Lion's mane, lutein, maca (Lepidium meyenii), mace, marjoram, matcha, milk thistle, mints (menthe), mulberry, Nardostachys chinensis, nutmeg, olive, oolong tea, orange (Citrus sinensis), oregano, papaya, paprika, pennyroyal, peppermint (Mentha piperita), pimento, potato peel, primrose, quercetin, quince, red clover, resveratrol, Rhizoma gastrodiae, Rhodiola, rooibos (red or green), rosehip (Rosa canina), rosemary, saffron, sage, Saint John's Wort, sandalwood, salvia (Salvia officinalis), savory, saw palmetto, Sceletium tortuosum, Schisandra, Silybum marianum, Skullcap, spearmint, Spikenard, spirulina, slippery elm bark, sorghum bran hi-tannin, sorghum grain hi-tannin, spearmint (Mentha spicata), spirulina, star anise, sumac bran, tarragon, thyme, tisanes, turmeric, Turnera aphrodisiaca, Uva ursi, valerian, vanilla, Viola odorata, white mulberry, wild yam root, wintergreen, withania somnifera, yacon root, yellow dock, yerba mate, and yerba santa

In some embodiments, the active ingredient comprises lemon balm. Lemon balm (Melissa officinalis) is a mildly lemon-scented herb from the same family as mint (Lamiaceae). The herb is native to Europe, North Africa, and West Asia. The tea of lemon balm, as well as the essential oil and the extract, are used in traditional and alternative medicine. In some embodiments, the active ingredient comprises lemon balm extract. In some embodiments, the lemon balm extract is present in an amount of from about 1 to about 4% by weight, based on the total weight of the composition.

In some embodiments, the active ingredient comprises ginseng. Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the ginseng is American ginseng or Korean ginseng. In specific embodiments, the active ingredient comprises Korean ginseng. In some embodiments, ginseng is present in an amount of from about 0.4 to about 0.6% by weight, based on the total weight of the composition.

### Stimulants

In some embodiments, the active ingredient comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis. In some embodiments, the active ingredient comprises caffeine. In some embodiments, the caffeine is present in an encapsulated form. On example of an encapsulated caffeine is Vitashure®, available from Balchem Corp., 52 Sunrise Park Road, New Hampton, NY, 10958.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition. In some embodiments, the composition comprises caffeine in an amount of from about 1.5 to about 6% by weight, based on the total weight of the composition;

### Amino Acids

In some embodiments, the active ingredient comprises an amino acid. As used herein, the term “amino acid” refers to an organic compound that contains amine (—NH₂) and carboxyl (—COOH) or sulfonic acid (SO₃H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, and beta-alanine. In some embodiments, the active ingredient comprises theanine. In some embodiments, the active ingredient comprises GABA. In some embodiments, the active ingredient comprises a combination of theanine and GABA. In some embodiments, the active ingredient is a combination of theanine, GABA, and lemon balm. In some embodiments, the active ingredient is a combination of caffeine, theanine, and ginseng. In some embodiments, the active ingredient comprises taurine. In some embodiments, the active ingredient is a combination of caffeine and taurine.

When present, an amino acid or combination of amino acids (e.g., theanine, GABA, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition.

### Vitamins

In some embodiments, the active ingredient comprises a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones). In some embodiments, the active ingredient comprises vitamin C. In some embodiments, the active ingredient is a combination of vitamin C, caffeine, and taurine.

When present, a vitamin or combination of vitamins (e.g., vitamin B6, vitamin B12, vitamin E, vitamin C, or a combination thereof) is typically at a concentration of from about 0.01% w/w to about 6% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, about 1%, about 2%, about 3%, about 4%, about 5%, or about 6% by weight, based on the total weight of the composition.

### Antioxidants

In some embodiments, the active ingredient comprises one or more antioxidants. As used herein, the term “antioxidant” refers to a substance which prevents or suppresses oxidation by terminating free radical reactions, and may delay or prevent some types of cellular damage. Antioxidants may be naturally occurring or synthetic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, Uva ursi, valerian, wild yam root, wintergreen, yacon root, yellow dock, yerba mate, yerba santa, bacopa monniera, withania somnifera, Lion's mane, and Silybum marianum. Such botanical materials may be provided in fresh or dry form, essential oils, or may be in the form of an extracts. The botanical materials (as well as their extracts) often include compounds from various classes known to provide antioxidant effects, such as minerals, vitamins, isoflavones, phytoesterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12 (2005) 216-220, which is incorporated herein by reference.

Non-limiting examples of other suitable antioxidants include citric acid, Vitamin E or a derivative thereof, a tocopherol, epicatechol, epigallocatechol, epigallocatechol gallate, erythorbic acid, sodium erythorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the total weight of the mixture/composition.

### Nicotine Component

In certain embodiments, the pouched products of the present disclosure can include a nicotinic compound. Various nicotinic compounds, and methods for their administration, are set forth in US Pat. Pub. No. 2011/0274628 to Borschke, which is incorporated herein by reference. As used herein, “nicotinic compound” or “source of nicotine” often refers to naturally-occurring or synthetic nicotinic compound unbound from a plant material, meaning the compound is at least partially purified and not contained within a plant structure, such as a tobacco leaf. Most preferably, nicotine is naturally-occurring and obtained as an extract from a Nicotiana species (e.g., tobacco). The nicotine can have the enantiomeric form S (−)-nicotine, R (+)-nicotine, or a mixture of S (−)-nicotine and R (+)-nicotine. Most preferably, the nicotine is in the form of S (−)-nicotine (e.g., in a form that is virtually all S (−)-nicotine) or a racemic mixture composed primarily or predominantly of S (−)-nicotine (e.g., a mixture composed of about 95 weight parts S (−)-nicotine and about 5 weight parts R (+)-nicotine). Most preferably, the nicotine is employed in virtually pure form or in an essentially pure form. Highly preferred nicotine that is employed has a purity of greater than about 95 percent, more preferably greater than about 98 percent, and most preferably greater than about 99 percent, on a weight basis.

In certain embodiments, a nicotine component may be included in the mixture in free base form, salt form, as a complex, or as a solvate. By “nicotine component” is meant any suitable form of nicotine (e.g., free base or salt) for providing oral absorption of at least a portion of the nicotine present. Typically, the nicotine component is selected from the group consisting of nicotine free base and a nicotine salt. In some embodiments, nicotine is in its free base form, which easily can be adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference.

In some embodiments, at least a portion of the nicotine can be employed in the form of a salt. Salts of nicotine can be provided using the types of ingredients and techniques set forth in U.S. Pat. No. 2,033,909 to Cox et al. and Perfetti, Beitrage Tabakforschung Int., 12:43-54 (1983), which are incorporated herein by reference. Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride. In some embodiments, the nicotine component or a portion thereof is a nicotine salt with one or more organic acids.

In some embodiments, at least a portion of the nicotine can be in the form of a resin complex of nicotine (“nicotine-polymer complex”). In some embodiments, the nicotine-polymer complex comprises a polymeric cation exchange resin which can comprise, e.g., a polyacrylic polymer. One non-limiting example of a suitable polymeric cation exchange resin is nicotine polacrilex, which is nicotine bound to, for example, a polymethacrilic acid, such as Amberlite IRP64, Purolite C115HMR, or Doshion P551. See, for example, U.S. Pat. No. 3,901,248 to Lichtneckert et al., which is incorporated herein by reference. Nicotine polacrilex is available in varying nicotine percentages, e.g., 18% to 20% nicotine; although not limited thereto, nicotine polacrilex generally comprises not less than 95% of the labeled amount of nicotine, calculated on an anhydrous basis. Another example is a nicotine-polyacrylic carbomer complex, such as with Carbopol 974P. The nicotine-polymer complex, where employed in embodiments of the disclosure, can be in the form of particles, which can be uncoated or can be coated, e.g., with one or more fillers and/or pH adjusters. The amount of nicotine-polymer complex, where included, can be up to about 20% by weight of the mixture, e.g., about 1% to about 20% by weight, about 1% to about 10% by weight, or about 4% to about 8% by weight. In some embodiments, the mixture disclosed herein comprises a nicotine component, wherein the nicotine component comprises a first and second nicotine component, wherein the first nicotine component is a nicotine-polymer complex and the second nicotine component is another form of nicotine (e.g., nicotine or a nicotine salt).

Typically, the nicotine component (calculated as the free base) when present, is in a concentration of at least about 0.001% by weight of the mixture, such as in a range from about 0.001% to about 10%. In some embodiments, the nicotine component is present in a concentration from about 0.1% w/w to about 10% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the mixture. In some embodiments, the nicotine component is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the mixture. These ranges can also apply to other active ingredients noted herein.

In some embodiments, the products or compositions of the disclosure can be characterized as free of any nicotine component (e.g., any embodiment as disclosed herein may be completely or substantially free of any nicotine component). By “substantially free” is meant that no nicotine has been intentionally added, beyond trace amounts that may be naturally present in e.g., a botanical material. For example, certain embodiments can be characterized as having less than 0.001% by weight of nicotine, or less than 0.0001%, or even 0% by weight of nicotine, calculated as the free base.

In some embodiments, the active ingredient comprises a nicotine component (e.g., any product or composition of the disclosure, in addition to comprising any active ingredient or combination of active ingredients as disclosed herein, may further comprise a nicotine component).

For customer satisfaction, it may be desirable to provide a basic amine-containing oral product configured for oral use which retains the initial basic amine content (e.g., nicotine content) during storage, and which delivers substantially the full amount of basic amine (e.g., nicotine) initially present in the oral product. In some such embodiments, nicotine or other basic amine is employed in association with at least a portion of an organic acid or an alkali metal salt thereof (referred to herein as “ion pairing”). Embodiments of the films disclosed herein can comprise at least one binder, a plasticizer, a basic amine, such as nicotine or a nicotine component; water; and an organic acid, an alkali metal salt of an organic acid, or a combination thereof, wherein the organic acid has a log P value of from about 1.4 to about 8.0.

As disclosed herein, at least a portion of the basic amine (e.g., nicotine) is associated with at least a portion of the organic acid or the alkali metal salt thereof. It is noted that for the purposes of the present disclosure, the basic amine can be included in place of or in addition to other active ingredients described in more detail herein. Depending on multiple variables (concentration, pH, nature of the organic acid, and the like), the basic amine present in the composition can exist in multiple forms, including ion paired, in solution (i.e., fully solvated), as the free base, as a cation, as a salt, or any combination thereof. The relative amounts of the various components within the oral product composition may vary, and typically are selected so as to provide the desired sensory and performance characteristics to the oral product. In some embodiments, the association between the basic amine and at least a portion of the organic acid or the alkali metal salt thereof is in the form of an ion pair between the basic amine and a conjugate base of the organic acid.

Ion pairing describes the partial association of oppositely charged ions in relatively concentrated solutions to form distinct chemical species called ion pairs. The strength of the association (i.e., the ion pairing) depends on the electrostatic force of attraction between the positive and negative ions (i.e., a protonated basic amine such as nicotine, and the conjugate base of the organic acid). By “conjugate base” is meant the base resulting from deprotonation of the corresponding acid (e.g., benzoate is the conjugate base of benzoic acid). On average, a certain population of these ion pairs exists at any given time, although the formation and dissociation of ion pairs is continuous. In the oral products as disclosed herein, and/or upon oral use of said oral products (e.g., upon contact with saliva), the basic amine, for example nicotine, and the conjugate base of the organic acid exist at least partially in the form of an ion pair. Without wishing to be bound by theory, it is believed that such ion pairing may minimize chemical degradation of the basic amine and/or enhance the oral availability of the basic amine (e.g., nicotine). At alkaline pH values (e.g., such as from about 7.5 to about 9), certain basic amines, for example nicotine, are largely present in the free base form, which has relatively low water solubility, and low stability with respect to evaporation and oxidative decomposition, but high mucosal availability. Conversely, at acidic pH values (such as from about 6.5 to about 4), certain basic amines, for example nicotine, are largely present in a protonated form, which has relatively high water solubility, and higher stability with respect to evaporation and oxidative decomposition, but low mucosal availability. Surprisingly, according to the present disclosure, it has been found that the properties of stability, solubility, and availability of the nicotine in a composition configured for oral use can be mutually enhanced through ion pairing or salt formation of nicotine with appropriate organic acids and/or their conjugate bases. Specifically, nicotine-organic acid ion pairs of moderate lipophilicity result in favorable stability and absorption properties. Lipophilicity is conveniently measured in terms of log P, the partition coefficient of a molecule between a lipophilic phase and an aqueous phase, usually octanol and water, respectively. An octanol-water partitioning favoring distribution of a basic amine-organic acid ion pair into octanol is predictive of good absorption of the basic amine present in the composition through the oral mucosa.

As noted above, at alkaline pH values (e.g., such as from about 7.5 to about 9), nicotine is largely present in the free base form (and accordingly, a high partitioning into octanol), while at acidic pH values (such as from about 6.5 to about 4), nicotine is largely present in a protonated form (and accordingly, a low partitioning into octanol). An ion pair between certain organic acids (e.g., having a log P value of from about 1.4 to about 8.0. such as from about 1.4 to about 4.5, allows nicotine partitioning into octanol consistent with that predicted for nicotine partitioning into octanol at a pH of 8.4.

One of skill in the art will recognize that the extent of ion pairing in the disclosed composition, both before and during use by the consumer, may vary based on, for example, pH, the nature of the organic acid, the concentration of nicotine, the concentration of the organic acid or conjugate base of the organic acid present in the composition, the moisture content of the composition, the ionic strength of the composition, and the like. One of skill in the art will also recognize that ion pairing is an equilibrium process influenced by the foregoing variables. Accordingly, quantification of the extent of ion pairing is difficult or impossible by calculation or direct observation. However, as disclosed herein, the presence of ion pairing may be demonstrated through surrogate measures such as partitioning of the nicotine between octanol and water or membrane permeation of aqueous solutions of the basic amine plus organic acids and/or their conjugate bases.

### Organic Acid

As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific composition ingredient as opposed to merely being inherently present as a component of another composition ingredient (e.g., the small amount of organic acid which may inherently be present in a composition ingredient, such as a tobacco material).

Suitable organic acids will typically have a range of lipophilicities (i.e., a polarity giving an appropriate balance of water and organic solubility). Typically, lipophilicities of suitable organic acids, as indicated by log P, will vary between about 1.4 and about 4.5 (more soluble in octanol than in water). In some embodiments, the organic acid has a log P value of from about 1.5 to about 4.0, e.g., from about 1.5, about 2.0, about 2.5, or about 3.0, to about 3.5, about 4.0, about 4.5, or about 5.0. Particularly suitable organic acids have a log P value of from about 1.7 to about 4, such as from about 2.0, about 2.5, or about 3.0, to about 3.5, or about 4.0. In specific embodiments, the organic acid has a log P value of about 2.5 to about 3.5. In some embodiments, organic acids outside this range may also be utilized for various purposes and in various amounts, as described further herein below. For example, in some embodiments, the organic acid may have a log P value of greater than about 4.5, such as from about 4.5 to about 8.0. Particularly, the presence of certain solvents or solubilizing agents (e.g., inclusion in the composition of glycerin or propylene glycol) may extend the range of lipophilicity (i.e., values of log P higher than 4.5, such as from about 4.5 to about 8.0).

Without wishing to be bound by theory, it is believed that moderately lipophilic organic acids (e.g., log P of from about 1.4 to about 4.5) produce ion pairs with nicotine which are of a polarity providing good octanol-water partitioning of the ion pair, and hence partitioning of nicotine, into octanol versus water. As discussed above, such partitioning into octanol is predictive of favorable oral availability. In some embodiments, the organic acid has a log P value of from about 1.4 to about 4.5, such as about 1.5, about 2, about 2.5, about 3, about 3.5, about 4 or about 4.5. In some embodiments, the organic acid has a log P value of from about 2.5 to about 3.5.

In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C₁-C₂₀). In some embodiments, the organic acid is an alkyl, cycloalkyl, heterocycloalkyl, aryl, or heteroaryl carboxylic or sulfonic acid.

As used herein, “alkyl” refers to any straight chain or branched chain hydrocarbon. The alkyl group may be saturated (i.e., having all sp3 carbon atoms), or may be unsaturated (i.e., having at least one site of unsaturation). As used herein, the term “unsaturated” refers to the presence of a carbon-carbon, sp2 double bond in one or more positions within the alkyl group. Unsaturated alkyl groups may be mono- or polyunsaturated. Representative straight chain alkyl groups include, but are not limited to, methyl, ethyl, n-propyl, n-butyl, n-pentyl, and n-hexyl. Branched chain alkyl groups include, but are not limited to, isopropyl, sec-butyl, isobutyl, tert-butyl, isopentyl, and 2-methylbutyl. Representative unsaturated alkyl groups include, but are not limited to, ethylene or vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. An alkyl group can be unsubstituted or substituted.

“Cycloalkyl” as used herein refers to a carbocyclic group, which may be mono- or bicyclic. Cycloalkyl groups include rings having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted, and may include one or more sites of unsaturation (e.g., cyclopentenyl or cyclohexenyl).

The term “aryl” as used herein refers to a carbocyclic aromatic group. Examples of aryl groups include, but are not limited to, phenyl and naphthyl. An aryl group can be unsubstituted or substituted.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl group comprises up to 20 carbon atoms and from 1 to 3 heteroatoms selected from N, O, and S. A heteroaryl or heterocycloalkyl may be a monocycle having 3 to 7 ring members (for example, 2 to 6 carbon atoms and 1 to 3 heteroatoms selected from N, O, and S) or a bicycle having 7 to 10 ring members (for example, 4 to 9 carbon atoms and 1 to 3 heteroatoms selected from N, O, and S), for example: a bicyclo[4,5], [5,5], [5,6], or [6,6] system. Examples of heteroaryl groups include by way of example and not limitation, pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl. Examples of heterocycloalkyls include by way of example and not limitation, dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl. Heteroaryl and heterocycloalkyl groups can be unsubstituted or substituted.

“Substituted” as used herein and as applied to any of the above alkyl, aryl, cycloalkyl, heteroaryl, heterocyclyl, means that one or more hydrogen atoms are each independently replaced with a substituent. Typical substituents include, but are not limited to, —Cl, Br, F, alkyl, —OH, —OCH₃, NH₂, —NHCH₃, —N(CH₃)₂, —CN, —NC(═O)CH₃, —C(═O)—, —C(═O)NH₂, and —C(═O)N(CH₃)₂. Wherever a group is described as “optionally substituted,” that group can be substituted with one or more of the above substituents, independently selected for each occasion. In some embodiments, the substituent may be one or more methyl groups or one or more hydroxyl groups.

In some embodiments, the organic acid is an alkyl carboxylic acid. Non-limiting examples of alkyl carboxylic acids include formic acid, acetic acid, propionic acid, butyric acid, valeric acid, caproic acid, heptanoic acid, octanoic acid, nonanoic acid, decanoic acid, undecanoic acid, dodecanoic acid, stearic acid, oleic acid, linoleic acid, linolenic acid, and the like.

In some embodiments, the organic acid is an alkyl sulfonic acid. Non-limiting examples of alkyl sulfonic acids include propanesulfonic acid, heptanesulfonic acid, and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like.

In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid.

Further non-limiting examples of organic acids which may be useful in certain embodiments include 2,2-dichloroacetic acid, 2-hydroxyethanesulfonic acid, 2-oxoglutaric acid, 4-acetamidobenzoic acid, 4-aminosalicylic acid, adipic acid, ascorbic acid (L), aspartic acid (L), alpha-methylbutyric acid, camphoric acid (+), camphor-10-sulfonic acid (+), cinnamic acid, cyclamic acid, dodecylsulfuric acid, ethane-1,2-disulfonic acid, ethanesulfonic acid, furoic acid, galactaric acid, gentisic acid, glucoheptonic acid, gluconic acid, glucuronic acid, glutamic acid, glycerophosphoric acid, glycolic acid, hippuric acid, isobutyric acid, isovaleric acid, lactobionic acid, lauric acid, levulinic acid, malic acid, malonic acid, mandelic acid, methanesulfonic acid, naphthalene-1,5-disulfonic acid, naphthalene-2-sulfonic acid, oleic acid, palmitic acid, pamoic acid, phenylacetic acid, pyroglutamic acid, pyruvic acid, sebacic acid, stearic acid, and undecylenic acid.

Examples of suitable acids include, but are not limited to, the list of organic acids in Table 1.

TABLE 1
Non-limiting examples of suitable organic acids

| Acid Name | log(P)* |
|---|---|
| benzoic acid | 1.9 |
| phenylacetic | 1.4 |
| p-toluic acid | 2.3 |
| ethyl benzoic acid | 2.9 |
| isopropyl benzoic acid | 3.5 |
| 4-phenylbutyric | 2.4 |
| 2-(4-Isobutylphenyl)propanoic acid | 3.5 |
| 2-napthoxyacetic acid | 2.5 |
| napthylacetic acid | 2.7 |
| heptanoic acid | 2.5 |
| octanoic acid | 3.05 |
| nonanoic acid | 3.5 |
| decanoic acid | 4.09 |
| 9-deceneoic acid | 3.3 |
| 2-deceneoic acid | 3.8 |
| 10-undecenoic acid | 3.9 |
| dodecandioic acid | 3.2 |
| dodecanoic acid | 4.6 |
| myristic acid | 5.3 |
| palmitic acid | 6.4 |
| stearic acid | 7.6 |
| cyclohexanebutanoic acid | 3.4 |
| 1-heptanesulfonic acid | 2.0 |
| 1-octanesulfonic acid | 2.5 |
| 1-nonanesulfonic acid | 3.1 |
| monooctyl succinate | 2.8 |
| tocopherol succinate | 10.2 |
| monomenthyl succinate | 3 |
| monomenthyl glutarate | 3.4 |
| norbixin ((2E,4E,6E,8E,10E,12E,14E,16E,18E)-4,8,13,17- | 7.2 |
| tetramethylicosa-2,4,6,8,10,12,14,16,18-nonaenedioic acid) | |
| bixin ((2E,4E,6E,8E,10E,12E,14E,16Z,18E)-20-methoxy- | 7.5 |
| 4,8,13,17-tetramethyl-20-oxoicosa-2,4,6,8,10,12,14,16,18- | |
| nonaenoic acid) | |

*Values obtained from PubChem or calculated

The selection of organic acid may further depend on additional properties in addition to consideration of the log P value. For example, an organic acid should be one recognized as safe for human consumption, and which has acceptable flavor, odor, volatility, stability, and the like. Determination of appropriate organic acids is within the purview of one of skill in the art.

In some embodiments, the organic acid is a mono ester of a dicarboxylic acid or a poly-carboxylic acid. In some embodiments, the dicarboxylic acid is malonic acid, succinic acid, glutaric acid, adipic acid, fumaric acid, maleic acid, or a combination thereof. In some embodiments, the dicarboxylic acid is succinic acid, glutaric acid, fumaric acid, maleic acid, or a combination thereof. In some embodiments, the dicarboxylic acid is succinic acid, glutaric acid, or a combination thereof.

In some embodiments, the alcohol forming the mono ester of the dicarboxylic acid is a lipophilic alcohol. Examples of suitable lipophilic alcohols include, but are not limited to, octanol, menthol, and tocopherol. In some embodiments, the organic acid is an octyl mono ester of a dicarboxylic acid, such as monooctyl succinate, monooctyl fumarate, or the like. In some embodiments, the organic acid is a monomenthyl ester of a dicarboxylic acid. Certain menthyl esters may be desirable in oral compositions as described herein by virtue of the cooling sensation they may provide upon use of the product comprising the composition. In some embodiments, the organic acid is monomenthyl succinate, monomenthyl fumarate, monomenthyl glutarate, or a combination thereof. In some embodiments, the organic acid is a monotocopheryl ester of a dicarboxylic acid. Certain tocopheryl esters may be desirable in oral compositions as described herein by virtue of the antioxidant effects they may provide. In some embodiments, the organic acid is tocopheryl succinate, tocopheryl fumarate, tocopheryl glutarate, or a combination thereof.

In some embodiments, the organic acid is a carotenoid derivative having one or more carboxylic acids. Carotenoids are tetraterpenes, meaning that they are produced from 8 isoprene molecules and contain 40 carbon atoms. Accordingly, they are usually lipophilic due to the presence of long unsaturated aliphatic chains, and are generally yellow, orange, or red in color. Certain carotenoid derivatives can be advantageous in oral compositions by virtue of providing both ion pairing and serving as a colorant in the composition. In some embodiments, the organic acid is 2E,4E,6E,8E,10E,12E,14E,16Z,18E)-20-methoxy-4,8,13,17-tetramethyl-20-oxoicosa-2,4,6,8,10,12,14,16,18-nonaenoic acid (bixin) or an isomer thereof. Bixin is an apocarotenoid found in annatto seeds from the achiote tree (Bixa orellana), and is the naturally occurring pigment providing the reddish orange color to annatto. Bixin is soluble in fats and alcohols but insoluble in water, and is chemically unstable when isolated, converting via isomerization into the double bond isomer, trans-bixin (β-bixin), having the structure:

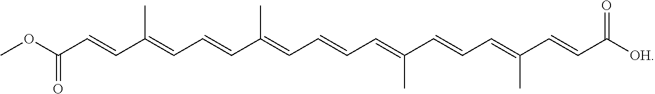 [SMILES: COC(=O)/C=C/C(C)=C/C=C/C(C)=C/C=C/C=C(C)/C=C/C=C(C)/C=C/C(=O)O]

In some embodiments, the organic acid is (2E,4E,6E,8E,10E,12E,14E,16E,18E)-4,8,13,17-tetramethylicosa-2,4,6,8,10,12,14,16,18-nonaenedioic acid (norbixin), a water soluble hydrolysis product of bixin having the structure:

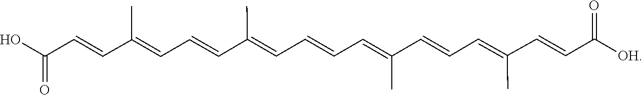 [SMILES: CC(/C=C/C=C(C)/C=C/C(=O)O)=C\C=C\C=C(C)\C=C\C=C(C)\C=C\C(=O)O]

In some embodiments, more than one organic acid may be present. For example, the composition may comprise two, or three, or four, or more organic acids. Accordingly, reference herein to “an organic acid” contemplates mixtures of two or more organic acids. The relative amounts of the multiple organic acids may vary. For example, a composition may comprise equal amounts of two, or three, or more organic acids, or may comprise different relative amounts. In this manner, it is possible to include certain organic acids (e.g., citric acid or myristic acid) which have a log P value outside the desired range, when combined with other organic acids to provide the desired average log P range for the combination. In some embodiments, it may be desirable to include organic acids in the composition which have log P values outside the desired range for purposes such as, but not limited to, providing desirable organoleptic properties, stability, as flavor components, and the like. Further, certain lipophilic organic acids have undesirable flavor and or aroma characteristics which would preclude their presence as the sole organic acid (e.g., in equimolar or greater quantities relative to nicotine). Without wishing to be bound by theory, it is believed that a combination of different organic acids may provide the desired ion pairing while the concentration of any single organic acid in the composition remains below the threshold which would be found objectionable from a sensory perspective.

In some embodiments, the composition comprises an organic acid which is a monoester of a dicarboxylic acid or is a carotenoid derivative having one or more carboxylic acids as described herein above, and further comprises an additional organic acid or salt thereof. In some embodiments, the additional organic acid is benzoic acid, an alkali metal salt thereof, or a combination thereof.

In some embodiments, the composition comprises an alkali metal salt of an organic acid. For example, at least a portion of the organic acid may be present in the composition in the form of an alkali metal salt. Suitable alkali metal salts include lithium, sodium, and potassium. In some embodiments, the alkali metal is sodium or potassium. In some embodiments, the alkali metal is sodium. In some embodiments, the composition comprises an organic acid and a sodium salt of the organic acid.

In some embodiments, the weight ratio of the organic acid to the sodium salt (or other alkali metal) of the organic acid is from about 0.1 to about 10, such as from about 0.1, about 0.25, about 0.3, about 0.5, about 0.75, or about 1, to about 2, about 5, or about 10. For example, in some embodiments, both an organic acid and the sodium salt thereof are added to the other components of the composition, wherein the organic acid is added in excess of the sodium salt, in equimolar quantities with the sodium salt, or as a fraction of the sodium salt. One of skill in the art will recognize that the relative amounts will be determined by the desired pH of the composition, as well as the desired ionic strength. For example, the organic acid may be added in a quantity to provide a desired pH level of the composition, while the alkali metal (e.g., sodium) salt is added in a quantity to provide the desired extent of ion pairing. As one of skill in the art will understand, the quantity of organic acid (i.e., the protonated form) present in the composition, relative to the alkali metal salt or conjugate base form present in the composition, will vary according to the pH of the composition and the pKa of the organic acid, as well as according to the actual relative quantities initially added to the composition.

The amount of organic acid or alkali metal salt thereof present in the composition, relative to the basic amine (e.g., nicotine), may vary. Generally, as the concentration of the organic acid (or the conjugate base thereof) increases, the percent of basic amine (e.g., nicotine) that is ion paired with the organic acid increases. This typically increases the partitioning of the basic amine (e.g., nicotine), in the form of an ion pair, into octanol versus water as measured by the log P (the log₁₀ of the partitioning coefficient). In some embodiments, the composition comprises from about 0.05, about 0.1, about 1, about 1.5, about 2, or about 5, to about 10, about 15, or about 20 molar equivalents of the organic acid, the alkali metal salt thereof, or the combination thereof, relative to the basic amine (e.g., nicotine), calculated as the free base of the basic amine.

In some embodiments, the composition comprises from about 2 to about 10, or from about 2 to about 5 molar equivalents of the organic acid, the alkali metal salt thereof, or the combination thereof, relative to the basic amine (e.g., nicotine), on a free-base basis. In some embodiments, the organic acid, the alkali metal salt thereof, or the combination thereof, is present in a molar ratio with basic amine (e.g., nicotine) from about 2, about 3, about 4, or about 5, to about 6, about 7, about 8, about 9, or about 10. In embodiments wherein more than one organic acid, alkali metal salt thereof, or both, are present, it is to be understood that such molar ratios reflect the totality of the organic acids present.

In certain embodiments the organic acid inclusion is sufficient to provide a composition pH of from about 4.0 to about 9.0, such as from about 4.5 to about 7.0, or from about 5.5 to about 7.0, from about 4.0 to about 5.5, or from about 7.0 to about 9.0. In some embodiments, the organic acid inclusion is sufficient to provide a composition pH of from about 4.5 to about 6.5, for example, from about 4.5, about 5.0, or about 5.5, to about 6.0, or about 6.5. In some embodiments, the organic acid is provided in a quantity sufficient to provide a pH of the composition of from about 5.5 to about 6.5, for example, from about 5.5, about 5.6, about 5.7, about 5.8, about 5.9, or about 6.0, to about 6.1, about 6.2, about 6.3, about 6.4, or about 6.5. In other embodiments, a mineral acid (e.g., hydrochloric acid, sulfuric acid, phosphoric acid, or the like) is added to adjust the pH of the composition to the desired value.

In some embodiments, the organic acid is added as the free acid, either neat (i.e., native solid or liquid form) or as a solution in, e.g., water, to the other composition components. In some embodiments, the alkali metal salt of the organic acid is added, either neat or as a solution in, e.g., water, to the other composition components. In some embodiments, the organic acid and the basic amine (e.g., nicotine) are combined to form a salt, either before addition to the composition, or the salt is formed within and is present in the composition as such. In other embodiments, the organic acid and basic amine (e.g., nicotine) are present as individual components in the composition, and form an ion pair upon contact with moisture (e.g., saliva in the mouth of the consumer).

In some embodiments, the organic acid is added as the free acid, either neat (i.e., native solid or liquid form) or as a solution in, e.g., water, to the other composition components. In some embodiments, the alkali metal salt of the organic acid is added, either neat or as a solution in, e.g., water, to the other composition components. In some embodiments, the organic acid and the basic amine (e.g., nicotine) are combined to form a salt, either before addition to the composition, or the salt is formed within and is present in the composition as such. In other embodiments, the organic acid and basic amine (e.g., nicotine) are present as individual components in the composition, and form an ion pair upon contact with moisture (e.g., saliva in the mouth of the consumer).

In some embodiments, the oral composition comprises nicotine benzoate and sodium benzoate, wherein at least a portion of the nicotine and benzoate ions present are in an ion paired form. In some embodiments, the composition comprises nicotine benzoate, sodium benzoate, and an organic acid, an alkali metal salt of an organic acid, or a combination thereof, the organic acid having a log P value from about 1 to about 12, wherein the organic acid is a monoester of a dicarboxylic acid or is a carotenoid derivative having one or more carboxylic acids.

In some embodiments, the oral composition further comprises a solubility enhancer to increase the solubility of one or more of the organic acid or salt thereof. Suitable solubility enhancers include, but are not limited to, humectants as described herein, such as glycerol or propylene glycol.

### Cannabinoids

In some embodiments, the active ingredient comprises one or more cannabinoids. As used herein, the term “cannabinoid” refers to a class of diverse chemical compounds that acts on cannabinoid receptors, also known as the endocannabinoid system, in cells that alter neurotransmitter release in the brain. Ligands for these receptor proteins include the endocannabinoids produced naturally in the body by animals; phytocannabinoids, found in cannabis; and synthetic cannabinoids, manufactured artificially. Cannabinoids found in cannabis include, without limitation: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), and tetrahydrocannabivarinic acid (THCV A). In certain embodiments, the cannabinoid is selected from tetrahydrocannabinol (THC), the primary psychoactive compound in cannabis, and/or cannabidiol (CBD) another major constituent of the plant, but which is devoid of psychoactivity. All of the above compounds can be used in the form of an isolate from plant material or synthetically derived.

In some embodiments, the cannabinoid (e.g., CBD) is added to the composition in the form of an isolate. An isolate is an extract from a plant, such as cannabis, where the active material of interest (in this case the cannabinoid, such as CBD) is present in a high degree of purity, for example greater than 95%, greater than 96%, greater than 97%, greater than 98%, or around 99% purity.

In some embodiments, the cannabinoid is an isolate of CBD in a high degree of purity, and the amount of any other cannabinoid in the composition is no greater than about 1% by weight of the composition, such as no greater than about 0.5% by weight of the composition, such as no greater than about 0.1% by weight of the composition, such as no greater than about 0.01% by weight of the composition. Alternatively, the active ingredient can be a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids.

When present, a cannabinoid (e.g., CBD) or cannabimimetic is typically in a concentration of at least about 0.1% by weight of the composition, such as in a range from about 0.1% to about 30%, such as, e.g., from about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 15%, about 20%, or about 30% by weight, based on the total weight of the composition. The choice of cannabinoid and the particular percentages thereof which may be present within the disclosed composition will vary depending upon the desired flavor, texture, and other characteristics of the composition.

### Terpenes

Active ingredients suitable for use in the present disclosure can also be classified as terpenes, many of which are associated with biological effects, such as calming effects. Terpenes are understood to have the general formula of (C5H8) n and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

In some embodiments, the terpene is a terpene derivable from a phytocannabinoid producing plant, such as a plant from the strain of the Cannabis sativa species, such as hemp. Suitable terpenes in this regard include so-called “C10” terpenes, which are those terpenes comprising 10 carbon atoms, and so-called “C15” terpenes, which are those terpenes comprising 15 carbon atoms. In some embodiments, the active ingredient comprises more than one terpene. For example, the active ingredient may comprise one, two, three, four, five, six, seven, eight, nine, ten or more terpenes as defined herein. In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

### Pharmaceutical Ingredients

In some embodiments, the active ingredient comprises an active pharmaceutical ingredient (API). The API can be any known agent adapted for therapeutic, prophylactic, or diagnostic use. These can include, for example, synthetic organic compounds, proteins and peptides, polysaccharides and other sugars, lipids, phospholipids, inorganic compounds (e.g., magnesium, selenium, zinc, nitrate), neurotransmitters or precursors thereof (e.g., serotonin, 5-hydroxytryptophan, oxitriptan, acetylcholine, dopamine, melatonin), and nucleic acid sequences, having therapeutic, prophylactic, or diagnostic activity. Non-limiting examples of APIs include analgesics and antipyretics (e.g., acetylsalicylic acid, acetaminophen, 3-(4-isobutylphenyl) propanoic acid), phosphatidylserine, myoinositol, docosahexaenoic acid (DHA, Omega-3), arachidonic acid (AA, Omega-6), S-adenosylmethionine (SAM), beta-hydroxy-beta-methylbutyrate (HMB), citicoline (cytidine-5′-diphosphate-choline), and cotinine. In some embodiments, the active ingredient comprises citicoline. In some embodiments, the active ingredient is a combination of citicoline, caffeine, theanine, and ginseng. In some embodiments, the active ingredient comprises sunflower lecithin. In some embodiments, the active ingredient is a combination of sunflower lecithin, caffeine, theanine, and ginseng.

The amount of API may vary. For example, when present, an API is typically at a concentration of from about 0.001% w/w to about 10% by weight, such as, e.g., from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the total weight of the composition.

In some embodiments, the composition is substantially free of any API. By “substantially free of any API” means that the composition does not contain, and specifically excludes, the presence of any API as defined herein, such as any Food and Drug Administration (FDA) approved therapeutic agent intended to treat any medical condition.

### Tobacco Material

In some embodiments, the mixture may include a tobacco material. The tobacco material can vary in species, type, and form. Generally, the tobacco material is obtained from for a harvested plant of the Nicotiana species. Example Nicotiana species include N. tabacum, N. rustica, N. alata, N. arentsii, N. excelsior, N. forgetiana, N. glauca, N. glutinosa, N. gossei, N. kawakamii, N. knightiana, N. langsdorffi, N. otophora, N. setchelli, N. sylvestris, N. tomentosa, N. tomentosiformis, N. undulata, N. x sanderae, N. africana, N. amplexicaulis, N. benavidesii, N. bonariensis, N. debneyi, N. longiflora, N. maritina, N. megalosiphon, N. occidentalis, N. paniculata, N. plumbaginifolia, N. raimondii, N. rosulata, N. simulans, N. stocktonii, N. suaveolens, N. umbratica, N. velutina, N. wigandioides, N. acaulis, N. acuminata, N. attenuata, N. benthamiana, N. cavicola, N. clevelandii, N. cordifolia, N. corymbosa, N. fragrans, N. goodspeedii, N. linearis, N. miersii, N. nudicaulis, N. obtusifolia, N. occidentalis subsp. Hersperis, N. pauciflora, N. petunioides, N. quadrivalvis, N. repanda, N. rotundifolia, N. solanifolia, and N. spegazzinii. Various representative other types of plants from the Nicotiana species are set forth in Goodspeed, The Genus Nicotiana, (Chonica Botanica) (1954); U.S. Pat. No. 4,660,577 to Sensabaugh, Jr. et al.; 5,387,416 to White et al., U.S. Pat. No. 7,025,066 to Lawson et al.; U.S. Pat. No. 7,798,153 to Lawrence, Jr. and 8,186,360 to Marshall et al.; each of which is incorporated herein by reference. Descriptions of various types of tobaccos, growing practices and harvesting practices are set forth in Tobacco Production, Chemistry and Technology, Davis et al. (Eds.) (1999), which is incorporated herein by reference.

Nicotiana species from which suitable tobacco materials can be obtained can be derived using genetic-modification or crossbreeding techniques (e.g., tobacco plants can be genetically engineered or crossbred to increase or decrease production of components, characteristics or attributes). See, for example, the types of genetic modifications of plants set forth in U.S. Pat. No. 5,539,093 to Fitzmaurice et al.; U.S. Pat. No. 5,668,295 to Wahab et al.; U.S. Pat. No. 5,705,624 to Fitzmaurice et al.; U.S. Pat. No. 5,844,119 to Weigl; U.S. Pat. No. 6,730,832 to Dominguez et al.; U.S. Pat. No. 7,173,170 to Liu et al.; U.S. Pat. No. 7,208,659 to Colliver et al. and 7,230,160 to Benning et al.; US Patent Appl. Pub. No. 2006/0236434 to Conkling et al.; and PCT WO2008/103935 to Nielsen et al. See, also, the types of tobaccos that are set forth in U.S. Pat. No. 4,660,577 to Sensabaugh, Jr. et al.; 5,387,416 to White et al.; and 6,730,832 to Dominguez et al., each of which is incorporated herein by reference.

The Nicotiana species can, in some embodiments, be selected for the content of various compounds that are present therein. For example, plants can be selected on the basis that those plants produce relatively high quantities of one or more of the compounds desired to be isolated therefrom. In certain embodiments, plants of the Nicotiana species (e.g., Galpao commun tobacco) are specifically grown for their abundance of leaf surface compounds. Tobacco plants can be grown in greenhouses, growth chambers, or outdoors in fields, or grown hydroponically.

Various parts or portions of the plant of the Nicotiana species can be included within a mixture as disclosed herein. For example, virtually all of the plant (e.g., the whole plant) can be harvested, and employed as such. Alternatively, various parts or pieces of the plant can be harvested or separated for further use after harvest. For example, the flower, leaves, stem, stalk, roots, seeds, and various combinations thereof, can be isolated for further use or treatment. In some embodiments, the tobacco material comprises tobacco leaf (lamina). The mixture disclosed herein can include processed tobacco parts or pieces, cured and aged tobacco in essentially natural lamina and/or stem form, a tobacco extract, extracted tobacco pulp (e.g., using water as a solvent), or a mixture of the foregoing (e.g., a mixture that combines extracted tobacco pulp with granulated cured and aged natural tobacco lamina).

In certain embodiments, the tobacco material comprises solid tobacco material selected from the group consisting of lamina and stems. The tobacco that is used for the mixture most preferably includes tobacco lamina, or a tobacco lamina and stem mixture (of which at least a portion is smoke-treated). Portions of the tobaccos within the mixture may have processed forms, such as processed tobacco stems (e.g., cut-rolled stems, cut-rolled-expanded stems or cut-puffed stems), or volume expanded tobacco (e.g., puffed tobacco, such as dry ice expanded tobacco (DIET)). See, for example, the tobacco expansion processes set forth in U.S. Pat. No. 4,340,073 to de la Burde et al.; U.S. Pat. No. 5,259,403 to Guy et al.; and 5,908,032 to Poindexter, et al.; and 7,556,047 to Poindexter, et al., all of which are incorporated by reference. In addition, the d mixture optionally may incorporate tobacco that has been fermented. See, also, the types of tobacco processing techniques set forth in PCT WO2005/063060 to Atchley et al., which is incorporated herein by reference.

The tobacco material is typically used in a form that can be described as particulate (i.e., shredded, ground, granulated, or powder form). The manner by which the tobacco material is provided in a finely divided or powder type of form may vary. Preferably, plant parts or pieces are comminuted, ground or pulverized into a particulate form using equipment and techniques for grinding, milling, or the like. Most preferably, the plant material is relatively dry in form during grinding or milling, using equipment such as hammer mills, cutter heads, air control mills, or the like. For example, tobacco parts or pieces may be ground or milled when the moisture content thereof is less than about 15 weight percent or less than about 5 weight percent. Most preferably, the tobacco material is employed in the form of parts or pieces that have an average particle size between 1.4 millimeters and 250 microns. In some instances, the tobacco particles may be sized to pass through a screen mesh to obtain the particle size range required. If desired, air classification equipment may be used to ensure that small sized tobacco particles of the desired sizes, or range of sizes, may be collected. If desired, differently sized pieces of granulated tobacco may be mixed together.

The manner by which the tobacco is provided in a finely divided or powder type of form may vary. Preferably, tobacco parts or pieces are comminuted, ground or pulverized into a powder type of form using equipment and techniques for grinding, milling, or the like. Most preferably, the tobacco is relatively dry in form during grinding or milling, using equipment such as hammer mills, cutter heads, air control mills, or the like. For example, tobacco parts or pieces may be ground or milled when the moisture content thereof is less than about 15 weight percent to less than about 5 weight percent. For example, the tobacco plant or portion thereof can be separated into individual parts or pieces (e.g., the leaves can be removed from the stems, and/or the stems and leaves can be removed from the stalk). The harvested plant or individual parts or pieces can be further subdivided into parts or pieces (e.g., the leaves can be shredded, cut, comminuted, pulverized, milled or ground into pieces or parts that can be characterized as filler-type pieces, granules, particulates or fine powders). The plant, or parts thereof, can be subjected to external forces or pressure (e.g., by being pressed or subjected to roll treatment). When carrying out such processing conditions, the plant or portion thereof can have a moisture content that approximates its natural moisture content (e.g., its moisture content immediately upon harvest), a moisture content achieved by adding moisture to the plant or portion thereof, or a moisture content that results from the drying of the plant or portion thereof. For example, powdered, pulverized, ground or milled pieces of plants or portions thereof can have moisture contents of less than about 25 weight percent, often less than about 20 weight percent, and frequently less than about 15 weight percent.

For the preparation of oral products, it is typical for a harvested plant of the Nicotiana species to be subjected to a curing process. The tobacco materials incorporated within the mixture for inclusion within products as disclosed herein are those that have been appropriately cured and/or aged. Descriptions of various types of curing processes for various types of tobaccos are set forth in Tobacco Production, Chemistry and Technology, Davis et al. (Eds.) (1999). Examples of techniques and conditions for curing flue-cured tobacco are set forth in Nestor et al., Beitrage Tabakforsch. Int., 20, 467-475 (2003) and U.S. Pat. No. 6,895,974 to Peele, which are incorporated herein by reference. Representative techniques and conditions for air curing tobacco are set forth in U.S. Pat. No. 7,650,892 to Groves et al.; Roton et al., Beitrage Tabakforsch. Int., 21, 305-320 (2005) and Staaf et al., Beitrage Tabakforsch. Int., 21, 321-330 (2005), which are incorporated herein by reference. Certain types of tobaccos can be subjected to alternative types of curing processes, such as fire curing or sun curing.

In certain embodiments, tobacco materials that can be employed include flue-cured or Virginia (e.g., K326), burley, sun-cured (e.g., Indian Kurnool and Oriental tobaccos, including Katerini, Prelip, Komotini, Xanthi and Yambol tobaccos), Maryland, dark, dark-fired, dark air cured (e.g., Madole, Passanda, Cubano, Jatin and Bezuki tobaccos), light air cured (e.g., North Wisconsin and Galpao tobaccos), Indian air cured, Red Russian and Rustica tobaccos, as well as various other rare or specialty tobaccos and various blends of any of the foregoing tobaccos.

The tobacco material may also have a so-called “blended” form. For example, the tobacco material may include a mixture of parts or pieces of flue-cured, burley (e.g., Malawi burley tobacco) and Oriental tobaccos (e.g., as tobacco composed of, or derived from, tobacco lamina, or a mixture of tobacco lamina and tobacco stem). For example, a representative blend may incorporate about 30 to about 70 parts burley tobacco (e.g., lamina, or lamina and stem), and about 30 to about 70 parts flue cured tobacco (e.g., stem, lamina, or lamina and stem) on a dry weight basis. Other example tobacco blends incorporate about 75 parts flue-cured tobacco, about 15 parts burley tobacco, and about 10 parts Oriental tobacco; or about 65 parts flue-cured tobacco, about 25 parts burley tobacco, and about 10 parts Oriental tobacco; or about 65 parts flue-cured tobacco, about 10 parts burley tobacco, and about 25 parts Oriental tobacco; on a dry weight basis. Other example tobacco blends incorporate about 20 to about 30 parts Oriental tobacco and about 70 to about 80 parts flue-cured tobacco on a dry weight basis.

Tobacco materials used in the present disclosure can be subjected to, for example, fermentation, bleaching, and the like. If desired, the tobacco materials can be, for example, irradiated, pasteurized, or otherwise subjected to controlled heat treatment. Such treatment processes are detailed, for example, in U.S. Pat. No. 8,061,362 to Mua et al., which is incorporated herein by reference. In certain embodiments, tobacco materials can be treated with water and an additive capable of inhibiting reaction of asparagine to form acrylamide upon heating of the tobacco material (e.g., an additive selected from the group consisting of lysine, glycine, histidine, alanine, methionine, cysteine, glutamic acid, aspartic acid, proline, phenylalanine, valine, arginine, compositions incorporating di- and trivalent cations, asparaginase, certain non-reducing saccharides, certain reducing agents, phenolic compounds, certain compounds having at least one free thiol group or functionality, oxidizing agents, oxidation catalysts, natural plant extracts (e.g., rosemary extract), and combinations thereof. See, for example, the types of treatment processes described in U.S. Pat. Nos. 8,434,496, 8,944,072, and 8,991,403 to Chen et al., which are all incorporated herein by reference. Further methods are disclosed, e.g., in Int. Pat. Appl. Pub. Nos. WO2013/122948; WO/2020/128971; WO/2021/048769; WO/2021/048768; WO/2021/048770; and Int. Pat. Appl. No. PCT/IB2021/058063, which are all incorporated herein by reference in their entireties. In certain embodiments, this type of treatment is useful where the original tobacco material is subjected to heat in the processes previously described.

In some embodiments, the type of tobacco material is selected such that it is initially visually lighter in color than other tobacco materials to some degree (e.g., whitened or bleached). Tobacco pulp can be whitened in certain embodiments according to any means known in the art. For example, bleached tobacco material produced by various whitening methods using various bleaching or oxidizing agents and oxidation catalysts can be used. Example oxidizing agents include peroxides (e.g., hydrogen peroxide), chlorite salts, chlorate salts, perchlorate salts, hypochlorite salts, ozone, ammonia, potassium permanganate, and combinations thereof. Example oxidation catalysts are titanium dioxide, manganese dioxide, and combinations thereof. Processes for treating tobacco with bleaching agents are discussed, for example, in U.S. Pat. Nos. 787,611 to Daniels, Jr.; 1,086,306 to Oelenheinz; U.S. Pat. No. 1,437,095 to Delling; U.S. Pat. No. 1,757,477 to Rosenhoch; U.S. Pat. No. 2,122,421 to Hawkinson; U.S. Pat. No. 2,148,147 to Baier; U.S. Pat. No. 2,170,107 to Baier; U.S. Pat. No. 2,274,649 to Baier; U.S. Pat. No. 2,770,239 to Prats et al.; U.S. Pat. No. 3,612,065 to Rosen; U.S. Pat. No. 3,851,653 to Rosen; U.S. Pat. No. 3,889,689 to Rosen; U.S. Pat. No. 3,943,940 to Minami; U.S. Pat. No. 3,943,945 to Rosen; U.S. Pat. No. 4,143,666 to Rainer; U.S. Pat. No. 4,194,514 to Campbell; U.S. Pat. Nos. 4,366,823, 4,366,824, and 4,388,933 to Rainer et al.; 4,641,667 to Schmekel et al.; U.S. Pat. No. 5,713,376 to Berger; U.S. Pat. No. 9,339,058 to Byrd Jr. et al.; 9,420,825 to Beeson et al.; and 9,950,858 to Byrd Jr. et al.; as well as in US Pat. App. Pub. Nos. 2012/0067361 to Bjorkholm et al.; 2016/0073686 to Crooks; 2017/0020183 to Bjorkholm; and 2017/0112183 to Bjorkholm, and in PCT Publ. Appl. Nos. WO1996/031255 to Giolvas and WO2018/083114 to Bjorkholm, all of which are incorporated herein by reference.

In some embodiments, the whitened tobacco material can have an ISO brightness of at least about 50%, at least about 60%, at least about 65%, at least about 70%, at least about 75%, or at least about 80%. In some embodiments, the whitened tobacco material can have an ISO brightness in the range of about 50% to about 90%, about 55% to about 75%, or about 60% to about 70%. ISO brightness can be measured according to ISO 3688:1999 or ISO 2470-1:2016.

In some embodiments, the whitened tobacco material can be characterized as lightened in color (e.g., “whitened”) in comparison to an untreated tobacco material. White colors are often defined with reference to the International Commission on Illumination's (CIE's) chromaticity diagram. The whitened tobacco material can, in certain embodiments, be characterized as closer on the chromaticity diagram to pure white than an untreated tobacco material.

In various embodiments, the tobacco material can be treated to extract a soluble component of the tobacco material therefrom. “Tobacco extract” as used herein refers to the isolated components of a tobacco material that are extracted from solid tobacco pulp by a solvent that is brought into contact with the tobacco material in an extraction process. Various extraction techniques of tobacco materials can be used to provide a tobacco extract and tobacco solid material. See, for example, the extraction processes described in US Pat. Appl. Pub. No. 2011/0247640 to Beeson et al., which is incorporated herein by reference. Other example techniques for extracting components of tobacco are described in U.S. Pat. No. 4,144,895 to Fiore; U.S. Pat. No. 4,150,677 to Osborne, Jr. et al.; 4,267,847 to Reid; U.S. Pat. No. 4,289,147 to Wildman et al.; U.S. Pat. No. 4,351,346 to Brummer et al.; 4,359,059 to Brummer et al.; U.S. Pat. No. 4,506,682 to Muller; U.S. Pat. No. 4,589,428 to Keritsis; U.S. Pat. No. 4,605,016 to Soga et al.; U.S. Pat. No. 4,716,911 to Poulose et al.; U.S. Pat. No. 4,727,889 to Niven, Jr. et al.; 4,887,618 to Bernasek et al.; U.S. Pat. No. 4,941,484 to Clapp et al.; U.S. Pat. No. 4,967,771 to Fagg et al.; U.S. Pat. No. 4,986,286 to Roberts et al.; U.S. Pat. No. 5,005,593 to Fagg et al.; U.S. Pat. No. 5,018,540 to Grubbs et al.; U.S. Pat. No. 5,060,669 to White et al.; U.S. Pat. No. 5,065,775 to Fagg; U.S. Pat. No. 5,074,319 to White et al.; U.S. Pat. No. 5,099,862 to White et al.; U.S. Pat. No. 5,121,757 to White et al.; U.S. Pat. No. 5,131,414 to Fagg; U.S. Pat. No. 5,131,415 to Munoz et al.; U.S. Pat. No. 5,148,819 to Fagg; U.S. Pat. No. 5,197,494 to Kramer; U.S. Pat. No. 5,230,354 to Smith et al.; U.S. Pat. No. 5,234,008 to Fagg; U.S. Pat. No. 5,243,999 to Smith; U.S. Pat. No. 5,301,694 to Raymond et al.; U.S. Pat. No. 5,318,050 to Gonzalez-Parra et al.; U.S. Pat. No. 5,343,879 to Teague; U.S. Pat. No. 5,360,022 to Newton; U.S. Pat. No. 5,435,325 to Clapp et al.; U.S. Pat. No. 5,445,169 to Brinkley et al.; U.S. Pat. No. 6,131,584 to Lauterbach; U.S. Pat. No. 6,298,859 to Kierulff et al.; U.S. Pat. No. 6,772,767 to Mua et al.; and 7,337,782 to Thompson, all of which are incorporated by reference herein.

Typical inclusion ranges for tobacco materials can vary depending on the nature and type of the tobacco material, and the intended effect on the final mixture (or composition), with an example range of up to about 30% by weight (or up to about 20% by weight or up to about 10% by weight or up to about 5% by weight), based on total weight of the mixture (e.g., about 0.1 to about 15% by weight). In some embodiments, a tobacco material (e.g., a whitened tobacco material) is included in a relatively small amount (e.g., about 0.01% to about 0.1% by weight).

In some embodiments, the products of the disclosure can be characterized as completely free or substantially free of tobacco material (other than purified nicotine which may optionally be included as an active ingredient). For example, certain embodiments can be characterized as having less than 1% by weight, or less than 0.5% by weight, or less than 0.1% by weight of tobacco material, or 0% by weight of tobacco material.

### Other Additives

Other additives can be included in the disclosed mixture (or composition). For example, the mixture can be processed, blended, formulated, combined and/or mixed with other materials or ingredients. The additives can be artificial, or can be obtained or derived from herbal or biological sources. Examples of further types of additives include thickening or gelling agents (e.g., fish gelatin), emulsifiers, preservatives (e.g., potassium sorbate and the like), zinc or magnesium salts selected to be relatively water soluble for compositions with greater water solubility (e.g., magnesium or zinc gluconate) or selected to be relatively water insoluble for compositions with reduced water solubility (e.g., magnesium or zinc oxide), disintegration aids, or combinations thereof. See, for example, those representative components, combination of components, relative amounts of those components, and manners and methods for employing those components, set forth in U.S. Pat. No. 9,237,769 to Mua et al., U.S. Pat. No. 7,861,728 to Holton, Jr. et al., US Pat. App. Pub. No. 2010/0291245 to Gao et al., and US Pat. App. Pub. No. 2007/0062549 to Holton, Jr. et al., each of which is incorporated herein by reference. Typical inclusion ranges for such additional additives can vary depending on the nature and function of the additive and the intended effect on the final mixture, with an example range of up to about 10% by weight, based on total weight of the mixture (e.g., about 0.1 to about 5% by weight).

The aforementioned additives can be employed together (e.g., as additive formulations) or separately (e.g., individual additive components can be added at different stages involved in the preparation of the final mixture). Furthermore, the aforementioned types of additives may be encapsulated as provided in the final product or mixture. Example encapsulated additives are described, for example, in WO2010/132444 to Atchley, which has been previously incorporated by reference herein.

### Particle Size Measurement

In some embodiments, any one or more of a filler component, a tobacco material, and the overall oral product described herein can be described as a particulate material. As used herein, the term “particulate” refers to a material in the form of a plurality of individual particles, some of which can be in the form of an agglomerate of multiple particles, wherein the particles have an average length to width ratio less than 2:1, such as less than 1.5:1, such as about 1:1. In various embodiments, the particles of a particulate material can be described as either substantially spherical or granular.

The particle size of a particulate material may be measured by sieve analysis, which provides particle size distribution data from which various particle size values can be determined, such as D10, D50, or D90. Particle size distribution data can also be used to calculate mean or average particle size, using methods such as set forth in ISO 9276-2:2001.

As the skilled person will readily appreciate, sieve analysis (otherwise known as a gradation test) is a method used to measure the particle size distribution of a particulate material. Typically, sieve analysis involves a nested column of sieves which comprise screens, preferably in the form of wire mesh cloths. A pre-weighed sample may be introduced into the top or uppermost sieve in the column, which has the largest screen openings or mesh size (i.e. the largest pore diameter of the sieve). Each lower sieve in the column has progressively smaller screen openings or mesh sizes than the sieve above. Typically, at the base of the column of sieves is a receiver portion to collect any particles having a particle size smaller than the screen opening size or mesh size of the bottom or lowermost sieve in the column (which has the smallest screen opening or mesh size).

In some embodiments, the column of sieves may be placed on or in a mechanical agitator. The agitator causes the vibration of each of the sieves in the column. The mechanical agitator may be activated for a pre-determined period of time in order to ensure that all particles are collected in the correct sieve. In some embodiments, the column of sieves is agitated for a period of time from 0.5 minutes to 10 minutes, such as from 1 minute to 10 minutes, such as from 1 minute to 5 minutes, such as for approximately 3 minutes. Once the agitation of the sieves in the column is complete, the material collected on each sieve is weighed. The weight of each sample on each sieve may then be divided by the total weight in order to obtain a percentage of the mass retained on each sieve. As the skilled person will readily appreciate, the screen opening sizes or mesh sizes for each sieve in the column used for sieve analysis may be selected based on the granularity or known maximum/minimum particle sizes of the sample to be analysed. In some embodiments, a column of sieves may be used for sieve analysis, wherein the column comprises from 2 to 20 sieves, such as from 5 to 15 sieves. In some embodiments, a column of sieves may be used for sieve analysis, wherein the column comprises 10 sieves. In some embodiments, the largest screen opening or mesh sizes of the sieves used for sieve analysis may be 1000 μm, such as 500 μm, such as 400 μm, such as 300 μm.

In some embodiments, any particulate material referenced herein (e.g., filler component, tobacco material, and the overall oral product) can be characterized as having at least 50% by weight of particles with a particle size as measured by sieve analysis of no greater than about 1000 μm, such as no greater than about 500 μm, such as no greater than about 400 μm, such as no greater than about 350 μm, such as no greater than about 300 μm. In some embodiments, at least 60% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 70% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 80% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 90% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 95% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 99% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, approximately 100% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm.

In some embodiments, at least 50% by weight, such as at least 60% by weight, such as at least 70% by weight, such as at least 80% by weight, such as at least 90% by weight, such as at least 95% by weight, such as at least 99% by weight of the particles of any particulate material referenced herein have a particle size as measured by sieve analysis of from about 0.01 μm to about 1000 μm, such as from about 0.05 μm to about 750 μm, such as from about 0.1 μm to about 500 μm, such as from about 0.25 μm to about 500 μm. In some embodiments, at least 50% by weight, such as at least 60% by weight, such as at least 70% by weight, such as at least 80% by weight, such as at least 90% by weight, such as at least 95% by weight, such as at least 99% by weight of the particles of any particulate material referenced herein have a particle size as measured by sieve analysis of from about 10 μm to about 400 μm, such as from about 50 μm to about 350 μm, such as from about 100 μm to about 350 μm, such as from about 200 μm to about 300 μm.

### Preparation of the Composition

The manner by which the various components of the mixture are combined may vary. As such, the overall mixture of various components with e.g., powdered mixture components, may be relatively uniform in nature. The components noted above, which may be in liquid or dry solid form, can be admixed in a pretreatment step prior to mixture with any remaining components of the mixture, or simply mixed together with all other liquid or dry ingredients. The various components of the mixture may be contacted, combined, or mixed together using any mixing technique or equipment known in the art. Any mixing method that brings the mixture ingredients into intimate contact can be used, such as a mixing apparatus featuring an impeller or other structure capable of agitation. Examples of mixing equipment include casing drums, conditioning cylinders or drums, liquid spray apparatus, conical-type blenders, ribbon blenders, mixers available as FKM130, FKM600, FKM1200, FKM2000 and FKM3000 from Littleford Day, Inc., Plough Share types of mixer cylinders, Hobart mixers, and the like. See also, for example, the types of methodologies set forth in U.S. Pat. No. 4,148,325 to Solomon et al.; U.S. Pat. No. 6,510,855 to Korte et al.; and 6,834,654 to Williams, each of which is incorporated herein by reference. In some embodiments, the components forming the mixture are prepared such that the mixture thereof may be used in a starch molding process for forming the mixture. Manners and methods for formulating mixtures will be apparent to those skilled in the art. See, for example, the types of methodologies set forth in U.S. Pat. No. 4,148,325 to Solomon et al.; U.S. Pat. No. 6,510,855 to Korte et al.; and U.S. Pat. No. 6,834,654 to Williams, U.S. Pat. No. 4,725,440 to Ridgway et al., and 6,077,524 to Bolder et al., each of which is incorporated herein by reference.

### Pouch Materials

In various embodiments, a moisture-permeable packet or pouch can act as a container for use of the composition within. For example, the pouch provides a liquid-permeable container of a type that may be considered to be similar in character to the mesh-like type of material that is used for the construction of a tea bag. If desired, flavoring ingredients, disintegration aids, and other desired components, may be incorporated within, or applied to, the pouch material. The composition/construction of such packets or pouches, such as the container pouch 20 in the embodiment illustrated in FIG. 1, may be varied as noted herein. For example, suitable packets, pouches or containers of the type used for the manufacture of smokeless tobacco products, which can be modified according to the present disclosure, are available under the tradenames CatchDry, Ettan, General, Granit, Goteborgs Rape, Grovsnus White, Metropol Kaktus, Mocca Anis, Mocca Mint, Mocca Wintergreen, Kicks, Probe, Prince, Skruf and TreAnkrare. A pouch type of product similar in shape and form to various embodiments of a pouched product described herein is commercially available as ZONNIC (distributed by Niconovum AB). Additionally, pouch type products generally similar in shape and form to various embodiments of a pouched product are set forth as snuff bag compositions E-J in Example 1 of PCT WO 2007/104573 to Axelsson et al., which is incorporated herein by reference, which are produced using excipient ingredients and processing conditions that can be used to manufacture pouched products as described herein.

The pouches can be formed from a fleece material, e.g., fibrous nonwoven webs. As used herein, the term “fiber” is defined as a basic element of textiles. Fibers are often in the form of a rope- or string-like element. As used herein, the term “fiber” is intended to include fibers, filaments, continuous filaments, staple fibers, and the like. The term “multicomponent fibers” refers to fibers that comprise two or more components that are different by physical or chemical nature, including bicomponent fibers. Specifically, the term “multicomponent fibers” includes staple and continuous fibers prepared from two or more polymers present in discrete structured domains in the fiber, as opposed to blends where the domains tend to be dispersed, random or unstructured.

A “fleece material” as used herein may be formed from various types of fibers (e.g., cellulosic fibers; such as viscose fibers, regenerated cellulose fibers, cellulose fibers, and wood pulps; cotton fibers; other natural fibers; or polymer/synthetic-type fibers) capable of being formed into a traditional fleece fabrics or other traditional pouch materials. For example, fleece materials may be provided in the form of a woven or nonwoven fabric. Suitable types of fleece materials, for example, are described in U.S. Pat. No. 8,931,493 to Sebastian et al.; US Patent App. Pub. No. 2016/0000140 to Sebastian et al.; and US Patent App. Pub. No. 2016/0073689 to Sebastian et al.; which are all incorporated herein by reference.

The term “nonwoven” is used herein in reference to fibrous materials, webs, mats, batts, or sheets in which fibers are aligned in an undefined or random orientation. The nonwoven fibers are initially presented as unbound fibers or filaments. An important step in the manufacturing of nonwovens involves binding the various fibers or filaments together. The manner in which the fibers or filaments are bound can vary, and include thermal, mechanical and chemical techniques that are selected in part based on the desired characteristics of the final product, as discussed in more detail below.

In various embodiments, the pouch material can be dissolvable (i.e., orally ingestible) such that under conditions of normal use (i.e., upon contact with saliva in the mouth of a user), the pouch material dissolves. Preferably, the pouch material will dissolve after a significant amount of the soluble components of the composition within the pouch (e.g., active ingredient(s) and/or flavorant(s)) permeate through the pouch material into the mouth of the user. For example, the pouch material can be configured to dissolve at a rate such that the pouch material holds the composition together for a period of time sufficient to allow for the release of substantially all water soluble components. As described herein, in certain embodiments, the composition within the pouch material can also be dissolvable. In such embodiments, the pouch material can be configured to dissolve at a rate similar to the rate at which the composition dissolves. In certain embodiments, the pouch material can be adapted to or configured to at least partially dissolve or completely dissolve in about 5 minutes or longer, about 15 minutes or longer, about 30 minutes or longer, or about an hour or longer. In certain embodiments, the pouch material can be adapted to or configured to at least partially dissolve or completely dissolve in no less than 30 minutes, no less than 45 minutes, or no less than an hour. In some embodiments, the pouch material may be adapted to or configured to at least partially dissolve or completely dissolve in a time of about 30 seconds to about 30 minutes, about 1 minute to about 25 minutes, about 5 minutes to about 20 minutes, or about 5 minutes to about 15 minutes. Without being limited by theory, a pouched product comprising a dissolvable pouch material can provide environmental advantages.

In various embodiments, dissolvable pouch materials can include, but are not limited to, spun or nonwoven alginate fibers, gluten fibers, mini-perforated flat sheets derived from alginate, carrageenan, and other polymer binders, and combinations thereof. Without being limited by theory, the dissolution rate of the pouch material can be controlled by the use of cross-linking technology between alginate or pectin and calcium salts, for example. In certain embodiments, the dissolvable pouch material can include fast dissolving fibers formed using an electrospinning process (e.g., solution-based electrospinning) with hydrophilic polymers. See, e.g., the techniques and fibers disclosed in Asawahame, Chawalinee et al., Formation of Orally Fast Dissolving Fibers Containing Propolis by Electrospinning Technique, Chiang Mai J. Sci. 2015; 42 (2), p. 469-480, which is herein incorporated by reference in its entirety.

In some embodiments, the fibers within the fleece material may include, but are not limited to, a polymer selected from the group consisting of polyglycolic acid, polylactic acid, polyhydroxyalkanoates, polycaprolactone, polybutylene succinate, polybutylene succinate adipate, and copolymers thereof. In some embodiments, the fibers within the fleece material may be selected from the groups consisting of wool, cotton, fibers made of cellulosic material, such as regenerated cellulose, cellulose acetate, cellulose triacetate, cellulose nitrate, ethyl cellulose, cellulose acetate propionate, cellulose acetate butyrate, hydroxypropyl cellulose, methyl hydroxypropyl cellulose, protein fibers, and the like. See also, the fiber types set forth in US Pat. Appl. Pub. No. 2014/0083438 to Sebastian et al., which is incorporated by reference herein. In various embodiments, the pouch material can include a polymer selected from the group consisting of polyvinylpyrrolidone, polyvinyl alcohol, and combinations thereof.

Regenerated cellulose fibers (e.g., viscose or lyocell fibers) can be particularly advantageous, and are typically prepared by extracting non-cellulosic compounds from wood, contacting the extracted wood with caustic soda, followed by carbon disulfide and then by sodium hydroxide, giving a viscous solution. The solution is subsequently forced through spinneret heads to create viscous threads of regenerated fibers. Example methods for the preparation of regenerated cellulose are provided in U.S. Pat. No. 4,237,274 to Leoni et al; U.S. Pat. No. 4,268,666 to Baldini et al; U.S. Pat. No. 4,252,766 to Baldini et al.; U.S. Pat. No. 4,388,256 to Ishida et al.; U.S. Pat. No. 4,535,028 to Yokogi et al.; U.S. Pat. No. 5,441,689 to Laity; U.S. Pat. No. 5,997,790 to Vos et al.; and U.S. Pat. No. 8,177,938 to Sumnicht, which are incorporated herein by reference. The manner in which the regenerated cellulose is made is not limiting, and can include, for example, both the rayon and the TENCEL processes. Various suppliers of regenerated cellulose are known, including Lenzing (Austria), Cordenka (Germany), Aditya Birla (India), and Daicel (Japan).

The fibers used in the nonwoven web according to the present disclosure can vary, and include fibers having any type of cross-section, including, but not limited to, circular, rectangular, square, oval, triangular, and multilobal. In certain embodiments, the fibers can have one or more void spaces, wherein the void spaces can have, for example, circular, rectangular, square, oval, triangular, or multilobal cross-sections. As noted previously, the fibers can be selected from single-component (i.e., uniform in composition throughout the fiber) or multicomponent fiber types including, but not limited to, fibers having a sheath/core structure and fibers having an islands-in-the-sea structure, as well as fibers having a side-by-side, segmented pie, segmented cross, segmented ribbon, or tipped multilobal cross-sections.

The physical parameters of the fibers present in the nonwoven web can vary. For example, the fibers used in the nonwoven web can have varying size (e.g., length, dpf) and crimp characteristics. In some embodiments, fibers used in the nonwoven web can be nano fibers, sub-micron fibers, and/or micron-sized fibers. In certain embodiments, fibers of the nonwoven webs useful herein can measure about 1.5 dpf to about 2.0 dpf, or about 1.6 dpf to about 1.90 dpf. In a preferred embodiment, each fiber can be a staple fiber. Each fiber length can measure about 35 mm to about 60 mm, or about 38 mm to about 55 mm, for example. In various embodiments, each fiber can measure about 4-10 crimps per cm, or about 5-8 crimps per cm. It can be advantageous for all fibers in the nonwoven web to have similar fiber size and crimp attributes to ensure favorable blending and orientation of the fibers in the nonwoven web.

The fibrous webs can have varying thicknesses, porosities and other parameters. The nonwoven web can be formed such that the fiber orientation and porosity of the pouched product formed therefrom can retain the composition adapted for oral use that is enclosed within the outer water-permeable pouch, but can also allow the flavors of the composition to be enjoyed by the consumer. For example, in some embodiments, the fibrous webs can have a basis weight of about 20 gsm to about 60 gsm, about 20 gsm to about 35 gsm, or about 25 gsm to about 30 gsm. In a preferred embodiment, the fibrous web can have a basis weight of about 28 gsm. Basis weight of a fabric can be measured using ASTM D3776/D3776M-09a (2013) (Standard Test Methods for Mass Per Unit Area (Weight) of Fabric), for example. In various embodiments, the fibrous web can have a thickness of about 0.1 mm to about 0.15 mm (e.g., about 0.11 mm). The fibrous web can have an elongation of about 70% to about 80%, e.g., about 78%. In some embodiments, the fibrous web can have a peak load of about 4 lbs. to about 8 lbs., e.g., about 5.5 lbs. Elongation and breaking strength of textile fabrics can be measured using ASTM D5034-09 (2013) (Standard Test Method for Breaking Strength and Elongation of Textile Fabrics (Grab Test)), for example. In various embodiments, the fibrous web can have a Tensile Energy Absorption (TEA) of about 35 to about 40, e.g., about 37. In certain embodiments, the fibrous web can have a porosity of greater than about 10,000 ml/min/cm2. TEA can be measured, for example, as the work done to break the specimen under tensile loading per lateral area of the specimen. Porosity, or air permeability of textile fabrics can be measured using ASTM D737-04 (2012) (Standard Test method for Air Permeability of Textile Fabrics), for example.

In various embodiments of the pouched product described herein, the outer water-permeable pouch is made from a nonwoven web as described above. In some embodiments, a pouch is constructed of a single layer of the nonwoven web. In various embodiments, the pouch material comprises a multilayer composite made up of two or more nonwoven layers, each layer being orally ingestible. Each nonwoven layer can be formed by processes discussed below. In a multilayer structure, a first layer can be relatively hydrophilic and a second layer can be relatively hydrophobic (compared to each other). In some embodiments, an outer water-permeable pouch can comprise an outer hydrophilic layer and an inner hydrophobic layer that can be in contact with the composition adapted for oral use. As such, the hydrophobic layer can, during storage of the pouched product, retain any moisture in the composition adapted for oral use such that flavors in the composition are not lost due to moisture loss. However, capillaries in the hydrophobic layer can wick out moisture into the mouth of the user, such that flavors are released into the oral cavity when used. In this manner, the pouch material can enhance storage stability without significantly compromising the enjoyment of the product by the end user. In less preferred embodiments, the relatively hydrophilic layer could be located on the interior of the multi-layer structure. The two layers can be formed into a multi-layer composite nonwoven material using any means known in the art, such as by attaching the two layers together using adhesive or stitching. The hydrophobicity of a textile material can be evaluated, for example, by measuring the contact angles between a drop of liquid and the surface of a textile material, as is known in the art.

In certain embodiments, the pouch material can comprise a flavor component which can be applied to the nonwoven layer in any conventional manner such as by coating, printing, and the like. In some embodiments of a pouched product described herein, the flavor within an outer pouch material can differ from a flavor contained within the internal composition adapted for oral use. For example, in certain embodiments, the pouch material can have a first flavor component and after the pouch material has dissolved, more moisture can reach the composition within the pouch material and a flavor component within the composition can be enhanced. In this manner, the product can be designed to provide multiple, different sensory experiences, a first sensory experience where the flavor in the outer pouch material transitions into the mouth of the user and a second sensory experience, typically occurring later in time, where the flavor of the internal composition transitions into the mouth of the user.

In some embodiments, a heat sealable binder coating or a binder material (e.g., a coating or other additive) may be added to the fibers prior to, during, or after forming the fleece material. As used herein, “heat sealable binder coatings” refers to coating materials, such as acrylic polymer compositions, applied to a substrate (e.g., a nonwoven web or fleece material) and which are capable of sealing seams of individual pouches upon heating. In some embodiments, a binder material can be added to the web fibers before or during the laying of the fibrous web (i.e., before the fibrous web is bonded to form a fleece material). In certain embodiments, a binder material can be added to the fleece material after it has been formed. In various embodiments, the binder material is in the form of a liquid coating. In certain embodiments, a binding powder can be applied to the fleece material. For example, powdered polyethylene can be used as a binder material. The liquid or powder coating can be applied, for example, between layers of fibers when cross-laying, air laying, or as an after treatment. A short exposure in an oven is sufficient to melt and fuse the binder material.

The means of producing the nonwoven web can vary. Web formation can be accomplished by any means known in the art. Web formation will typically involve a carding step, which involves deposition of the fibers onto a surface followed by aligning/blending the fibers in a machine direction. Thereafter, the fibrous web is typically subjected to some type of bonding/entanglement including, but not limited to, thermal fusion or bonding, mechanical entanglement, chemical adhesive, or a combination thereof. In one embodiment, the fibrous web is bonded thermally using a calendar (which can provide flat or point bonding), steam jet bonding, or a thru-air oven. Additional bonding methods include ultrasonic bonding and crimping. In some embodiments, needle punching is utilized, wherein needles are used to provide physical entanglement between fibers. In one embodiment, the web is entangled using hydroentanglement, which is a process used to entangle and bond fibers using hydrodynamic forces. As noted above, a binder material can be applied to the fibers of the fibrous web before laying the fibrous web, during formation of the fibrous web, and/or after the fibrous web has been bonded to form a fleece material. After forming the fleece material, heat can be applied to the fleece material in order to activate/at least partially melt the binder material to further bond the fleece material and thereby further enhance the mechanical integrity of the fleece material.

Methods for forming a nonwoven web comprising natural and synthetic fibers may include drylaid, airlaid and wetlaid methods. In some embodiments, the nonwoven fabric can be formed using a spunlaid or spunmelt process, which includes both spunbond and meltblown processes, wherein such processes are understood to typically entail melting, extruding, collecting and bonding thermoplastic polymer materials to form a fibrous nonwoven web. The technique of meltblowing is known in the art and is discussed in various patents, for example, U.S. Pat. No. 3,849,241 to Butin, U.S. Pat. No. 3,987,185 to Buntin et al., U.S. Pat. No. 3,972,759 to Buntin, and 4,622,259 to McAmish et al., each of which is herein incorporated by reference in its entirety. General spunbonding processes are described, for example, in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, and 30 3,542,615 to Dobo et al., which are all incorporated herein by reference.

In various embodiments, the nonwoven web is made by providing a dry laid or a spun laid web of fibers, and then needle punching the web to bond the dry laid or spun laid web. The needle punched fleece material is produced when barbed needles are pushed through the fibrous web, forcing some fibers upwards or downwards through the web by the barbed needles. The fibers punched through the web remain at their new position once the needles are withdrawn. This needling action interlocks fibers and holds the structure together by inter fiber friction forces caused by compression of the web, thereby bonding the web. By displacing a sufficient number of fibers in the web, the web is converted into a nonwoven fabric.

In certain embodiments, the nonwoven web is made by a fleece carding process with point bonding. The point bonding (e.g., using a calendar) should be limited to a relatively small portion of the surface area of the nonwoven web to maintain good porosity in the web for migration of water-soluble components through the web during oral use. In certain embodiments, the point bonding is limited to less than about 60% of the surface area of the nonwoven web (or resulting pouch), such as less than about 50%, less than about 30%, or less than about 20% (e.g., about 1% to about 50%, about 5% to about 40%, or about 10% to about 30%). An advantage of point bonding is the ability to control the porosity, flexibility and fabric strength.

In other embodiments, the nonwoven web can be subjected to hydroentangling. The term “hydroentangled” or “spunlaced” as applied to a nonwoven fabric herein defines a web subjected to impingement by a curtain of high speed, fine water jets, typically emanating from a nozzle jet strip accommodated in a pressure vessel often referred to as a manifold or an injector. This hydroentangled fabric can be characterized by reoriented, twisted, turned and entangled fibers. For example, the fibers can be hydroentangled by exposing the nonwoven web to water pressure from one or more hydroentangling manifolds at a water pressure in the range of about 10 bar to about 1000 bar. As compared to point bonding, spunlace technology, in certain embodiments, will have less impact on porosity of the web and, thus, may enhance flavor transfer through the nonwoven pouch material.

In various embodiments, the nonwoven web can be subjected to a second bonding method in order to reduce elongation of the web during processing. In certain embodiments, nonwoven webs of the present disclosure can exhibit significant elongation during high speed processing on pouching equipment. Too much elongation of the nonwoven web can cause the web to shrink during processing, such that the final product is not sized appropriately. As such, it can be necessary to modify process equipment to fit a wider roll of fleece, for example, to compensate for any shrinkage in the final product due to elongation.

In order to avoid or at least reduce such an elongation problem, in various embodiments the nonwoven web can be point bonded after the first bonding (e.g., hydroentangling) is completed. A second bonding process can increase the tensile strength of the nonwoven web and reduce elongation characteristics. In particular, a point bonding process can bond a nonwoven web by partially or completely melting the web (e.g., the heat sealable binder material) at discrete points. For example, in some embodiments, the nonwoven web can be subjected to ultrasonic bonding after initial bonding of the web. Any ultrasonic bonding system for nonwoven materials known in the art can be used to ultrasonically bond the nonwoven web. See, for example, the apparatuses and devices disclosed in U.S. Pat. No. 8,096,339 to Aust and 8,557,071 to Weiler, incorporated by reference herein. In some embodiments, the nonwoven web can be subjected to point bonding via embossed and/or engraved calendar rolls, which are typically heated. See, e.g., the point bonding methods incorporating the use of very high calendar pressures and embossing techniques discussed in U.S. Pat. Publ. No. 2008/0249492 to Schmidt, herein incorporated by reference in its entirety. The point bonding process is typically limited to less than about 60% of the surface area of the nonwoven web as noted above.

In certain embodiments, the processing techniques used to blend, entangle and bond the nonwoven web can also impart a desired texture to the fibrous nonwoven web material. For instance, point bonding or hydroentangling can impart a desired texture (e.g. a desired pattern) to the nonwoven web. This textured pattern can include product identifying information. In some embodiments, the product identifying information is selected from the group consisting of product brand, a company name, a corporate logo, a corporate brand, a marketing message, product strength, active ingredient, product manufacture date, product expiration date, product flavor, product release profile, weight, product code (e.g., batch code), other product differentiating markings, and combinations thereof.

### Preparation of Pouched Products

Various manufacturing apparatuses and methods can be used to create a pouched product described herein. For example, US Publication No. 2012/0055493 to Novak, III et al., incorporated by reference in its entirety, relates to an apparatus and process for providing pouch material formed into a tube for use in the manufacture of smokeless tobacco products. The pouch material can include a binder material according to the present disclosure (e.g., a binder material comprising an aliphatic polyester). Similar apparatuses that incorporate equipment for supplying a continuous supply of a pouch material (e.g., a pouch processing unit adapted to supply a pouch material to a continuous tube forming unit for forming a continuous tubular member from the pouch material) can be used to create a pouched product described herein. Representative equipment for forming such a continuous tube of pouch material is disclosed, for example, in U.S. Patent Application Publication No. US 2010/0101588 to Boldrini et al., which is incorporated herein by reference in its entirety. The apparatus further includes equipment for supplying pouched material to the continuous tubular member such that, when the continuous tubular member is subdivided and sealed into discrete pouch portions, each pouch portion includes a charge of a composition adapted for oral use. Representative equipment for supplying the filler material is disclosed, for example, in U.S. Patent Application Publication No. US 2010/0018539 to Brinkley, which is incorporated herein by reference in its entirety. In some instances, the apparatus may include a subdividing unit for subdividing the continuous tubular member into individual pouch portions and, once subdivided into the individual pouch portions, may also include a sealing unit for sealing at least one of the ends of each pouch portion. In other instances, the continuous tubular member may be sealed into individual pouch portions with a sealing unit and then, once the individual pouch portions are sealed, the continuous tubular member may be subdivided into discrete individual pouch portions by a subdividing unit subdividing the continuous tubular member between the sealed ends of serially-disposed pouch portions. Still in other instances, sealing (closing) of the individual pouch portions of the continuous tubular member may occur substantially concurrently with the subdivision thereof, using a closing and dividing unit.

An example apparatus for manufacturing an oral pouch product is illustrated in FIGS. 1-5 of U.S. Publication No. 2012/0055493 to Novak, III et al.; however, this apparatus is used in a generic and descriptive sense only and not for purposes of limitation. It should also be appreciated that the following manufacturing process and related equipment is not limited to the process order described below. In various embodiments of the present disclosure, an apparatus similar to that described in U.S. Publication No. 2012/0055493 can be configured to removably receive a first bobbin on an unwind spindle assembly, the first bobbin having a continuous length of a material, such as a pouch material, wound thereon. When the first bobbin is engaged with the apparatus, the pouch material can be routed from the first bobbin to a forming unit configured to form a continuous supply of the pouch material into a continuous tubular member defining a longitudinal axis.

As such, as the pouch material is unwound from the first bobbin, the pouch material can be directed around an arrangement of roller members, otherwise referred to herein as a dancer assembly. A forming unit can be configured to cooperate with the first bobbin and the dancer assembly to take up slack in the pouch material and to maintain a certain amount of longitudinal tension on the pouch material as the pouch material is unwound from the first bobbin and fed to the forming unit, for example, by a drive system. One of ordinary skill in the art will appreciate that, between the first bobbin and the forming unit, the pouch material can be supported, routed, and/or guided by a suitably aligned series of any number of, for example, idler rollers, guideposts, air bars, turning bars, guides, tracks, tunnels, or the like, for directing the pouch material along the desired path. Typical bobbins used by conventional automated pouch making apparatuses often contain a continuous strip of pouch material of which the length may vary. As such, the apparatus described herein can be configured so as to handle bobbins of that type and size.

The forming unit can include one or more roller members configured to direct the pouch material about a hollow shaft such that the continuous supply of the pouch material can be formed into a continuous tubular member. The forming unit can include a sealing device configured to seal, fix, or otherwise engage lateral edges of the pouch material to form a longitudinally-extending seam, thereby forming a longitudinally-extending continuous tubular member. In various embodiments, an insertion unit can be configured to introduce charges of the composition adapted for oral use into the continuous tubular member through the hollow shaft. The insertion unit may be directly or indirectly engaged with the hollow shaft.

A leading edge or end (also referred to as a laterally-extending seam) of the continuous tubular member can be closed/sealed such that a charge of composition adapted for oral use inserted by the insertion unit, is contained within the continuous tubular member proximate to the leading end. The leading end can be closed/sealed via a closing and dividing unit configured to close/seal a first portion of the continuous tubular member to form the closed leading end of a pouch member portion. The closing and dividing unit can also be configured to form a closed trailing edge or end of a previous pouch member portion. In this regard, the closing and dividing unit can also be configured to close a second portion of the continuous tubular member to form the closed trailing end of the pouch member portion. In this regard, the closing and dividing unit can close the ends, by heat-sealing, or other suitable sealing mechanism.

As illustrated in FIGS. 20-22 of U.S. Publication No. 2012/0055493 to Novak, III et al., the closing and dividing unit can be configured to divide the continuous tubular member, between the closed trailing end and the closed leading end of serially-disposed pouch member portions, along the longitudinal axis of the continuous tubular member, and into a plurality of discrete pouch member portions such that each discrete pouch member portion includes a portion of the oral composition from the insertion unit. In this regard, the closing and dividing unit can include a blade, heated wire, or other cutting arrangement for severing the continuous tubular member into discrete pouch member portions. For example, the closing and dividing unit can include first and second arm members configured to interact to close and divide the continuous tubular member.

In operation, a charge of the composition adapted for oral use (i.e., an amount suitable for an individual pouch member portion) can be supplied to the pouch member portion by an insertion unit after a leading end has been closed, but prior to the closing of a trailing end. In various embodiments, after receiving the charge of the oral composition, the discrete individual pouch member portion can be formed by closing the trailing end and severing the closed pouch member portion from the continuous tubular member such that an individual pouched product is formed.

The amount of material contained within each pouch may vary. In various embodiments, the weight of the mixture within each pouch is at least about 50 mg, for example, from about 50 mg to about 2 grams, from about 100 mg to about 1.5 grams, or from about 200 mg to about 700 mg. In certain smaller embodiments, the dry weight of the material within each pouch is at least about 50 mg to about 150 mg. For some larger embodiment, the dry weight of the material within each pouch preferably does not exceed about 300 mg to about 500 mg. In some embodiments, each pouch/container may have disposed therein a flavor agent member, as described in greater detail in U.S. Pat. No. 7,861,728 to Holton, Jr. et al., which is incorporated herein by reference. For example, at least one flavored strip, piece or sheet of flavored water dispersible or water soluble material (e.g., a breath-freshening edible film type of material) may be disposed within each pouch along with or without at least one capsule. Such strips or sheets may be folded or crumpled in order to be readily incorporated within the pouch. See, for example, the types of materials and technologies set forth in U.S. Pat. No. 6,887,307 to Scott et al. and 6,923,981 to Leung et al.; and The EFSA Journal (2004) 85, 1-32; which are incorporated herein by reference.

In various embodiments, the nonwoven web can be sufficiently tacky so as to create issues with high-speed pouching equipment. Therefore, in certain embodiments, a Teflon coating, or similar material, can be applied to one or more surfaces of the pouching equipment that touch the nonwoven web such as, for example, rollers, cutting instruments, and heat sealing devices in order to reduce and/or alleviate any problems associated with the pouch material sticking to the pouching equipment during processing.

The pouched products can further include product identifying information printed or dyed on the outer water-permeable pouch or imprinted (e.g., embossed, debossed, or otherwise pressed) on the outer water-permeable pouch, such as described in U.S. Pat. Appl. Pub. No. 2014/0255452 to Reddick et al., filed Mar. 11, 2013, which is incorporated by reference herein. As noted above, flavorants can also be incorporated into the nonwoven web if desired, such as by coating or printing an edible flavorant ink onto the nonwoven web. See, e.g., U.S. Pat. Appl. Pub. Nos. 2012/0085360 to Kawata et al. and 2012/0103353 to Sebastian et al., each of which is herein incorporated by reference.

A pouched product as described herein can be packaged within any suitable inner packaging material and/or outer container. See also, for example, the various types of containers for smokeless types of products that are set forth in U.S. Pat. No. 7,014,039 to Henson et al.; U.S. Pat. No. 7,537,110 to Kutsch et al.; U.S. Pat. No. 7,584,843 to Kutsch et al.; U.S. Pat. No. 8,397,945 to Gelardi et al., D592,956 to Thiellier; D594,154 to Patel et al.; and D625,178 to Bailey et al.; US Pat. Pub. Nos. 2008/0173317 to Robinson et al.; 2009/0014343 to Clark et al.; 2009/0014450 to Bjorkholm; 2009/0250360 to Bellamah et al.; 2009/0266837 to Gelardi et al.; 2009/0223989 to Gelardi; 2009/0230003 to Thiellier; 2010/0084424 to Gelardi; and 2010/0133140 to Bailey et al; 2010/0264157 to Bailey et al.; and 2011/0168712 to Bailey et al. which are incorporated herein by reference.

Products of the present disclosure configured for oral use may be packaged and stored in any suitable packaging in much the same manner that conventional types of smokeless tobacco products are packaged and stored. For example, a plurality of packets or pouches may be contained in a cylindrical container. The storage period of the product after preparation may vary. As used herein, “storage period” refers to the period of time after the preparation of the disclosed product. In some embodiments, one or more of the characteristics of the products disclosed herein (e.g., retention of whiteness, lack of color change, retention of volatile flavor components) is exhibited over some or all of the storage period. In some embodiments, the storage period (i.e., the time period after preparation) is at least one day. In some embodiments, the storage period is from about about 1 day, about 2 days, or about 3 days, to about 1 week, or from about 1 week to about 2 weeks, from about 2 weeks to about 1 month, from about 1 month to about 2 months, from about 2 months to about 3 months, from about 3 months to about 4 months, or from about 4 months to about 5 months. In some embodiments, the storage period is any number of days between about 1 and about 150. In certain embodiments, the storage period may be longer than 5 months, for example, about 6 months, about 7 months, about 8 months, about 9 months, about 10 months, about 11 months, or about 12 months.

### EXPERIMENTAL

Several nonwoven pouched products were produced, containing a mixture comprising substantially spherical MCC, according to the following Examples. Reference to 100 micron MCC spheres refers to Vivapur® 100 MCC spheres. Reference to 200 micron MCC spheres refers to Vivapur® 200 MCC spheres. Reference to 350 micron MCC spheres refers to Vivapur® 350 MCC spheres. Reference to 500 micron MCC spheres refers to Vivapur® 500 MCC Spheres. Reference to 700 micron MCC spheres refers to Vivapur® 700 MCC spheres. Reference to 1000 micron MCC spheres refers to Vivapur® 1000 MCC spheres.

### Nicotine Dissolution Testing

Certain pouched product examples below were evaluated for nicotine release versus time. Pouched samples (1 pouch for each replicate) were accurately weighed and added to a dissolution vessel containing 500 ml of dissolution medium (12 mM ammonium phosphate, pH 7.4) at 37° C. The dissolution vessel was transferred to the dissolution apparatus and samples collected according to the parameters in Table 2.

Nicotine content of the samples was determined via high performance liquid chromatography (HPLC) analysis using Waters Acquity H-class system (or equivalent) with UV detection. HPLC conditions and program are listed in Tables 3 and 4. The experimental sample response was plotted against a linear calibration curve generated with standards of known concentration to accurately determine the nicotine content in the samples.

TABLE 2
Dissolution parameters

| | |
|---|---|
| Apparatus | 708-DS Apparatus1, Baskets |
| Sampling Station | 850-DS |
| Shaft Speed | 50 RPM (0 to 60 min) |
| | 250 RPM (60 to 90 min) |
| Dissolution Temperature | 37.0 ± 0.5° C. |
| Dissolution Volume | 500 mL |
| Sampling Time Points | 1,3.5,6,8.5,11,15,20,30,60 min (50 |
| | RPM) 90 min (250 RPM, Infinity) |
| Sample Collection Volume | 1.5 mL |
| Pull volume | 2.0 mL (1.5 mL sample + 0.5 mL waste |
| | drop) |
| Filter Plate | 8-channel 0.45 μm Nylon Filter |

TABLE 3
HPLC Conditions

| | |
|---|---|
| Mobile Phase A | 60/40 Phosphate Buffer |
| | (25 mM pH 7.2)/MeOH |
| Mobile Phase B | MeOH |
| Wavelength (UV) | 259 nm |
| Column Set Point Temperature | 35 ± 1° C. |
| Autosampler Temperature | 6 ± 4° C. |
| Flow Rate | 0.75 mL/min |
| Injection Volume | 10 μL |
| HPLC Mode | Gradient (see Table 5) |
| Run Time | 7.0 min |

TABLE 4
HPLC Program

| | Flow Rate | | |
|---|---|---|---|
| Time (min) | (ml/min) | % A | % B |
| Initial | 0.75 | 100 | 0 |
| 2.1 | 0.75 | 100 | 0 |
| 2.5 | 0.75 | 0 | 100 |
| 4.5 | 0.75 | 0 | 100 |
| 5.0 | 0.75 | 100 | 0 |
| 7.0 | 0.75 | 100 | 0 |

### Example 1: MCC Sphere-Containing Composition Comprising Nicotine Polacrilex

A pouched product was prepared, comprising a mixture within a pouch, wherein the composition of the mixture within the pouch is provided in Table 5 below (with all values provided in weight percentages based on the total weight of the mixture plus additional added water). The MCC spheres were about 500 μm in average diameter (with a particle size distribution between 500 μm and 710 μm). The components were combined and a given amount of the mixture (e.g., about 500 mg or about 700 mg) was placed within a non-woven fleece pouch. The oven volatile content of the mixture was about 15%. Neither pouch was sprayed with additional water, i.e., the final pouch weights were about 500 mg and about 700 mg, respectively. The nicotine content of each final pouch (based on the weight of each pouch) was about 0.6% and about 0.8% respectively by weight.

TABLE 5
MCC Sphere-Containing Composition
Comprising Nicotine Polacrilex

| Ingredient | Amount | |
|---|---|---|
| MCC spheres | 60%-90% | |
| Sodium chloride | 1%-5% | |
| Xylitol | 1%-5% | |
| Sucralose | 0.01% | to 0.5% |
| Acesulfame-K | 0.01% | to 0.5% |
| Nicotine polacrilex | 1%-5% | |
| Water | 1% | to 8% |
| Glycerin | 2% | to 10% |
| Flavor | 0.1% | to 1% |
| Sodium hydroxide | 1% | to 5% |

### Example 2: MCC Sphere-Containing Composition Comprising Nicotine Extract

A pouched product was prepared, comprising a mixture within a pouch, wherein the composition of the mixture within the pouch is provided in Table 6 below (with all values provided in weight percentages based on the total weight of the mixture plus additional added water). The MCC spheres were about 500 μm in average diameter (with a particle size distribution between 500 μm and 710 μm). The components were combined and a given amount of the mixture (e.g., about 500 mg or about 700 mg) was placed within a non-woven fleece pouch. The oven volatile content of the mixture was about 15%. Neither pouch was sprayed with additional water, i.e., the final pouch weights were about 500 mg and about 700 mg, respectively. The nicotine content of each final pouch (based on the weight of each pouch) was about 0.6% and about 0.8% respectively by weight.

TABLE 6
MCC Sphere-Containing Composition Comprising Nicotine Extract

| Ingredient | Amount | |
|---|---|---|
| MCC spheres | 60%-90% | |
| Sodium chloride | 1%-5% | |
| Xylitol | 1%-5% | |
| Sucralose | 0.01% | to 0.5% |
| Acesulfame-K | 0.01% | to 0.5% |
| Nicotine extract | 2%-8% | |
| Water | 1% | to 8% |
| Glycerin | 2% | to 10% |
| Flavor | 0.1% | to 1% |
| Citric Acid | 0.5% | to 2% |

### Example 3: Nicotine Dissolution Data for Examples 1 and 2

Pouches made using the general recipes of Examples 1 and 2 above were subjected to nicotine dissolution testing as set forth above. The results are shown in FIG. 3, which compares the nicotine dissolution characteristics of Example 1 and Example 2 to pouched products containing conventional particulate, non-spherical MCC. As shown, both Example 1 and Example 2 exhibited significantly faster nicotine release/dissolution as compared to the conventional MCC products.

### Example 4: Additional Pouch Examples

A series of additional pouch products were made, each with the recipe set forth in the tables below using the same general manufacturing method noted in Examples 1 and 2.

### Pouch AA (Control):

TABLE 7
Pouch AA Composition

| Ingredient | % Inclusion |
|---|---|
| Particulate MCC | 55.16% |
| Sodium Chloride | 2.12% |
| Water | 7.39% |
| 12% Nicotine solution | 8.39% |
| Propylene Glycol | 1.00% |
| Xylitol | 1.63% |
| Sucralose | 0.28% |
| Flavor | 1.62% |
| Water Overspray | 16.00% |
| Fleece | 6.41% |
| | 100.00% |

TABLE 8
Pouch AA Characteristics

| | |
|---|---|
| Fill Moisture (Water, PG, Flavor) | 27% |
| Water (mg, fill) | 80.48 |
| Water (mg, overspray) | 94.08 |
| Pouch Moisture (Water) | 34% |
| Calculated Oven Volatile Content | 37% |
| Flavor (mg, pouch) | 9.5 |
| Nicotine (mg, pouch) | 5.9 |

### Pouch AB:

TABLE 9
Pouch AB Composition

| Ingredient | % Inclusion |
|---|---|
| 350 Micron MCC Spheres | 63.67% |
| Sodium Chloride | 2.11% |
| Water | 0.77% |
| 12% Nicotine solution | 9.55% |
| Propylene Glycol | 1.12% |
| Xylitol | 1.61% |
| Sucralose | 0.28% |
| Flavor | 1.61% |
| Water Overspray | 12.00% |
| Fleece | 7.28% |
| | 100.00% |

TABLE 10
Pouch AB Characteristics

| | |
|---|---|
| Fill Moisture (Water, PG, Flavor) | 19% |
| Water (mg, fill) | 41.09 |
| Water (mg, overspray) | 62.16 |
| Pouch Moisture (Water) | 25% |
| Calculated Oven Volatile Content | 28% |
| Flavor (mg, pouch) | 8.3 |
| Nicotine (mg, pouch) | 5.9 |

### Pouch AC

TABLE 11
Pouch AC Composition

| Ingredient | % Inclusion |
|---|---|
| 350 Micron MCC Spheres | 64.81% |
| Sodium Chloride | 2.11% |
| Water | 4.97% |
| 12% Nicotine solution | 4.77% |
| Propylene Glycol | 0.56% |
| Xylitol | 1.61% |
| Sucralose | 0.28% |
| Flavor | 1.61% |
| Water Overspray | 12.00% |
| Fleece | 7.28% |
| | 100.00% |

TABLE 12
Pouch AC Characteristics

| | |
|---|---|
| Fill Moisture (Water, PG, Flavor) | 19% |
| Water (mg, fill) | 44.30 |
| Water (mg, overspray) | 62.16 |
| Pouch Moisture (Water) | 25% |
| Calculated Oven Volatile Content | 27% |
| Flavor (mg, pouch) | 8.3 |
| Nicotine (mg, pouch) | 3.0 |

### Pouch AE:

TABLE 13
Pouch AE Composition

| Ingredient | % Inclusion |
|---|---|
| 500 Micron MCC Spheres | 64.81% |
| Sodium Chloride | 2.11% |
| Water | 4.97% |
| 12% Nicotine solution | 4.77% |
| Propylene Glycol | 0.56% |
| Xylitol | 1.61% |
| Sucralose | 0.28% |
| Flavor | 1.61% |
| Water Overspray | 12.00% |
| Fleece | 7.28% |
| | 100.00% |

TABLE 14
Pouch AE Characteristics

| | |
|---|---|
| Fill Moisture (Water, PG, Flavor) | 19% |
| Water (mg, fill) | 44.30 |
| Water (mg, overspray) | 62.16 |
| Pouch Moisture (Water) | 25% |
| Calculated Oven Volatile Content | 27% |
| Flavor (mg, pouch) | 8.3 |
| Nicotine (mg, pouch) | 3.0 |

### Pouch AG:

TABLE 15
Pouch AG Composition

| Ingredient | % Inclusion |
|---|---|
| 350 Micron MCC Spheres | 66.34% |
| Sodium Chloride | 2.12% |
| 25% Nicotine solution | 2.30% |
| Glycerin | 10.00% |
| Xylitol | 1.62% |
| Sucralose | 0.28% |
| Flavor | 1.38% |
| Water Overspray | 8.69% |
| Fleece | 7.28% |
| | 100.00% |

TABLE 16
Pouch AG Characteristics

| | |
|---|---|
| Fill Moisture (Water, VG, Flavor) | 20% |
| Water (mg, fill) | 8.93 |
| Water (mg, overspray) | 45.03 |
| Pouch Moisture (Water) | 14% |
| Calculated Oven Volatile Content | 26% |
| Flavor (mg, pouch) | 7.2 |
| Nicotine (mg, pouch) | 3.0 |

### Pouch AH:

TABLE 17
Pouch AH Composition

| Ingredient | % Inclusion |
|---|---|
| 350 Micron MCC Spheres | 66.21% |
| Sodium Chloride | 2.13% |
| 25% Nicotine solution | 2.31% |
| Propylene Glycol | 5.00% |
| Glycerin | 10.00% |
| Xylitol | 1.63% |
| Sucralose | 0.28% |
| Flavor | 1.39% |
| Water Overspray | 3.79% |
| Fleece | 7.28% |
| | 100.00% |

TABLE 18
Pouch AH Characteristics

| | |
|---|---|
| Fill Moisture (Water, PG, VG, Flavor) | 25% |
| Water (mg, fill) | 8.97 |
| Water (mg, overspray) | 19.63 |
| Pouch Moisture (Water) | 9% |
| Calculated Oven Volatile Content | 26% |
| Flavor (mg, pouch) | 7.2 |
| Nicotine (mg, pouch) | 3.0 |

### Pouch AI:

TABLE 19
Pouch AI Composition

| Ingredient | % Inclusion |
|---|---|
| Particulate MCC | 23.48% |
| Sodium Chloride | 2.11% |
| Water | 4.97% |
| 12% Nicotine solution | 4.77% |
| Propylene Glycol | 0.56% |
| Xylitol | 1.61% |
| Sucralose | 0.28% |
| 350 Micron MCC Spheres | 41.33% |
| Flavor | 1.61% |
| Water Overspray | 12.00% |
| Fleece | 7.28% |
| | 100.00% |

TABLE 20
Pouch AI Characteristics

| | |
|---|---|
| Fill Moisture (Water, PG, Flavor) | 16% |
| Water (mg, fill) | 44.30 |
| Water (mg, overspray) | 62.16 |
| Pouch Moisture (Water) | 23% |
| Calculated Oven Volatile Content | 25% |
| Flavor (mg, pouch) | 8.3 |
| Nicotine (mg, pouch) | 3.0 |

### Pouch AJ:

TABLE 21
Pouch AJ Composition

| Ingredient | % Inclusion |
|---|---|
| Particulate MCC | 37.04% |
| Sodium Chloride | 2.11% |
| Water | 4.97% |
| 12% Nicotine solution | 4.77% |
| Propylene Glycol | 0.56% |
| Xylitol | 1.61% |
| Sucralose | 0.28% |
| 700 Micron MCC Spheres | 27.77% |
| Flavor | 1.61% |
| Water Overspray | 12.00% |
| Fleece | 7.28% |
| | 100.00% |

TABLE 22
Pouch AJ Characteristics

| | |
|---|---|
| Fill Moisture (Water, PG, Flavor) | 17% |
| Water (mg, fill) | 44.30 |
| Water (mg, overspray) | 62.16 |
| Pouch Moisture (Water) | 23% |
| Calculated Oven Volatile Content | 26% |
| Flavor (mg, pouch) | 8.3 |
| Nicotine (mg, pouch) | 3.0 |

### Pouch AK:

TABLE 23
Pouch AK Composition

| Ingredient | % Inclusion |
|---|---|
| Particulate MCC | 46.19% |
| Sodium Chloride | 2.11% |
| Water | 4.97% |
| 12% Nicotine solution | 4.77% |
| Propylene Glycol | 0.56% |
| Xylitol | 1.61% |
| Sucralose | 0.28% |
| 1000 Micron MCC Spheres | 18.62% |
| Flavor | 1.61% |
| Water Overspray | 12.00% |
| Fleece | 7.28% |
| | 100.00% |

TABLE 24
Pouch AK Characteristics

| | |
|---|---|
| Fill Moisture (Water, PG, Flavor) | 17% |
| Water (mg, fill) | 44.30 |
| Water (mg, overspray) | 62.16 |
| Pouch Moisture (Water) | 24% |
| Calculated Oven Volatile Content | 26% |
| Flavor (mg, pouch) | 8.3 |
| Nicotine (mg, pouch) | 3.0 |

### Pouch AN:

TABLE 25
Pouch AN Composition

| Ingredient | % Inclusion |
|---|---|
| Particulate MCC | 16.38% |
| Sodium Chloride | 2.11% |
| Water | 4.97% |
| 12% Nicotine solution | 4.77% |
| Propylene Glycol | 0.56% |
| Xylitol | 1.61% |
| Sucralose | 0.28% |
| 500 Micron MCC Spheres | 48.43% |
| Flavor | 1.61% |
| Water Overspray | 12.00% |
| Fleece | 7.28% |
| | 100.00% |

TABLE 26
Pouch AN Characteristics

| | |
|---|---|
| Fill Moisture (Water, PG, Flavor) | 15% |
| Water (mg, fill) | 44.30 |
| Water (mg, overspray) | 62.16 |
| Pouch Moisture (Water) | 22% |
| Calculated Oven Volatile Content | 24% |
| Flavor (mg, pouch) | 8.3 |
| Nicotine (mg, pouch) | 3.0 |

### Pouch AO:

TABLE 27
Pouch AO Composition

| Ingredient | % Inclusion |
|---|---|
| 350 Micron MCC Spheres | 64.81% |
| Sodium Chloride | 2.11% |
| Water | 2.46% |
| 12% Nicotine solution | 2.29% |
| Propylene Glycol | 0.56% |
| Xylitol | 1.61% |
| Sucralose | 0.28% |
| Glycerin | 5.00% |
| Flavor | 1.61% |
| Water Overspray | 12.00% |
| Fleece | 7.28% |
| | 100.00% |

TABLE 28
Pouch AO Characteristics

| | |
|---|---|
| Fill Moisture (Water, PG, Flavor) | 13% |
| Water (mg, fill) | 21.63 |
| Water (mg, overspray) | 62.16 |
| Pouch Moisture (Water) | 20% |
| Calculated Oven Volatile Content | 27% |
| Flavor (mg, pouch) | 8.3 |
| Nicotine (mg, pouch) | 3.0 |

### Pouch AP:

TABLE 29
Pouch AP Composition

| Ingredient | % Inclusion |
|---|---|
| 350 Micron MCC Spheres | 64.25% |
| Sodium Chloride | 2.11% |
| 12% Nicotine solution | 2.30% |
| Propylene Glycol | 0.56% |
| Xylitol | 1.62% |
| Sucralose | 0.28% |
| Glycerin | 10.00% |
| Flavor | 1.61% |
| Water Overspray | 10.00% |
| Fleece | 7.28% |
| | 100.00% |

TABLE 30
Pouch AP Characteristics

| | |
|---|---|
| Fill Moisture (Water, PG, Flavor) | 9% |
| Water (mg, fill) | 8.92 |
| Water (mg, overspray) | 51.80 |
| Pouch Moisture (Water) | 16% |
| Calculated Oven Volatile Content | 28% |
| Flavor (mg, pouch) | 8.3 |
| Nicotine (mg, pouch) | 3.0 |

### Example 5: Nicotine Dissolution Data for Selected Pouches of Example 4

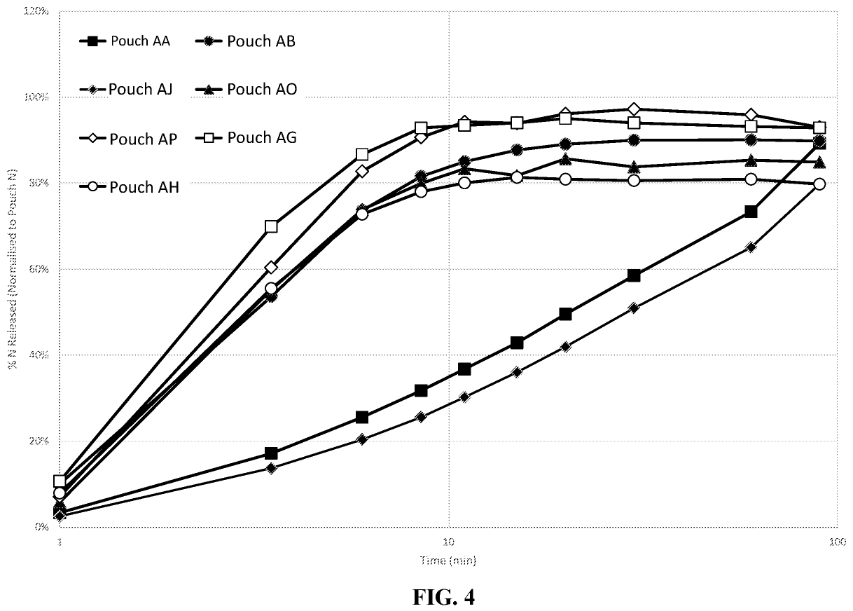

Pouches AA (control), AB, AG, AH, AJ, AO, and AP were subjected to nicotine dissolution testing as set forth above. The results are shown in FIG. 4. As shown, all pouches with MCC spheres only (no conventional particulate MCC) exhibited significantly faster nicotine release/dissolution as compared to the control. The faster nicotine dissolution was seen in samples with both higher nicotine and water levels (e.g., AB) and at lower nicotine and water levels (e.g., AO and AP). The fastest nicotine dissolution was seen in samples with higher glycerin levels (e.g., AG and AP), particularly in the relative absence of propylene glycol.

### Example 6: Sensory Evaluation for Selected Pouches of Example 4

Pouches AA (control), AB, AC, AG, AJ, AO, and AP were subjected to a sensory panel to evaluate flavor intensity and time to flavor release on a scale of +3 as compared to the control. Each pouch was evaluated by four or five panelists. The results indicated that all of the samples containing MCC spheres (AB, AC, AG, AJ, AO, AP) exhibited quicker flavor release as compared to the control (AA). In addition, all of the samples other than AG exhibited more intense flavor as compared to the control. This suggests that inclusion of MCC spheres leads to quicker, more intense flavor release as compared to a sample containing only conventional particulate MCC.

### Example 7: Additional Pouch Examples with Sphere Coating or Ion Pairing

A series of additional pouch products were made, each with the recipe set forth in the tables below using the same general manufacturing method noted in Examples 1 and 2.

### Pouch AA′:

TABLE 31
Pouch AA′ Composition

| Ingredient | % Inclusion |
|---|---|
| 350 Micron MCC Spheres | 78.55 |
| Sodium Chloride | 3.15 |
| Xylitol | 1.76 |
| Sucralose | 0.15 |
| Ace-k | 0.07 |
| Sodium Bicarbonate | 0.26 |
| 12% Nicotine solution | 8.89 |
| Water | 0.87 |
| Glycerin | 6.00 |
| Flavor | 0.30 |
| | 100.00% |

TABLE 31
Pouch AA′ Characteristics

| | |
|---|---|
| Water (mg, overspray) | 0.00 |
| Calculated Oven Volatile Content | 15% |
| Nicotine (mg, pouch) | 5.73 |

### Pouch AB′:

TABLE 32
Pouch AB′ Composition

| Ingredient | % Inclusion |
|---|---|
| 350 Micron MCC Spheres | 75.91 |
| Sodium Chloride | 3.15 |
| Xylitol | 1.76 |
| Sucralose | 0.15 |
| Ace-k | 0.07 |
| Sodium Bicarbonate | 0.00 |
| 12% Nicotine solution | 8.89 |
| Benzoic Acid | 0.81 |
| Sodium Benzoate | 2.08 |
| Water | 0.87 |
| Glycerin | 6.00 |
| Flavor | 0.30 |
| | 100.00% |

TABLE 33
Pouch AB′ Characteristics

| | |
|---|---|
| Water (mg, overspray) | 0.00 |
| Calculated Oven Volatile Content | 15% |
| Nicotine (mg, pouch) | 5.73 |

### Pouch AC′:

TABLE 34
Pouch AC′ Composition

| Ingredient | % Inclusion |
|---|---|
| Particulate MCC | 36.94 |
| Sodium Chloride | 2.30 |
| Coated MCC Spheres* | 24.33 |
| (Nicotine/Pea Starch coating) | |
| Water | 14.00 |
| Propylene Glycol | 0.60 |
| Sodium bicarbonate | 0.26 |
| Xylitol | 1.76 |
| Sucralose | 0.30 |
| Flavor | 1.50 |
| Water Overspray | 18.00 |
| | 100.00% |

*Coating was about 15% by weight, based on weight of coated sphere, and comprised nicotine (2.87% by weight of coating), water (6% by weight of coating), with the balance being hydroxypropyl pea starch

TABLE 35
Pouch AC′ Characteristics

| | |
|---|---|
| Calculated Oven Volatile Content | 32% |
| Nicotine (mg, pouch) | 3.96 |

### Example 8: Nicotine Dissolution Data for Selected Pouches of Example 7

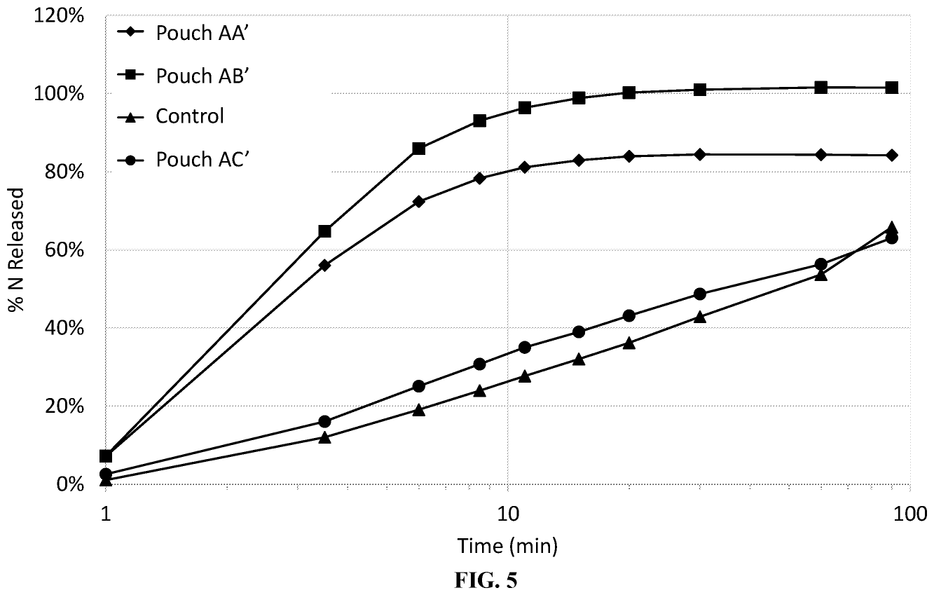

Pouches AA′, AB′, and AC′ of Example 7 were subjected to nicotine dissolution testing as set forth above. A control pouch sample containing conventional particulate MCC, 32% total moisture, and 4 mg of nicotine was also prepared and tested. The results are shown in FIG. 5. The pouches containing only MCC spheres (AA′ and AB′) again showed much faster nicotine dissolution as compared to the control.

### Example 9: Nicotine Dissolution Data Correlated to MCC Sphere Size

A selection of pouches were made with varying nicotine source and varying MCC sphere size to determine the impact of nicotine source and sphere size on nicotine dissolution. The tested pouches contained MCC spheres, sodium chloride, sodium bicarbonate, xylitol, sucralose, Ace-k, water, propylene glycol, and flavor, in addition to either a 12% nicotine solution or nicotine polacrilex. The target moisture for each pouch was about 11% by weight and the target nicotine amount (calculated as free base) was 4.0 mg per pouch. The pouches were otherwise identical except for the variance in nicotine source and sphere size. Control samples containing conventional particulate MCC were also prepared. Each pouch was subjected to nicotine dissolution testing as set forth above. The results are shown in Table 36 below, which are normalized to 100% nicotine dissolution at 90 minutes.

As shown, both pouches containing nicotine polacrilex and a 12% nicotine solution in combination with MCC spheres showed greater nicotine dissolution rates as compared to the control samples. In addition, the data shows that nicotine dissolution rates (particularly from the 7 minute data point through the 20 minute data point) for samples comprising the mid-sized spheres were greater than the smaller or larger sphere size samples, suggesting there is a mid-sized range of sphere size that provides optimal nicotine dissolution characteristics, such as a sphere size in the range of 350 to 650 microns in average particle size or D50 (median) particle size.

TABLE 36
Nicotine Dissolution Data

| Sample | Nicotine | MCC | | | | | | | | |
|---|---|---|---|---|---|---|---|---|---|---|
| Name | Source | Source | 3 min | 7 min | 11 min | 15 min | 20 min | 30 min | 60 min | 90 min |
| 093021-1 | Polacrilex | 200 μm | 1.20% | 50.60% | 68.60% | 76.30% | 82.30% | 86.40% | 92.60% | 100.00% |
| | | spheres | | | | | | | | |
| 093021-2 | Polacrilex | 350 μm | 7.10% | 56.90% | 73.80% | 80.40% | 84.90% | 89.70% | 95.10% | 100.00% |
| | | spheres | | | | | | | | |
| 093021-3 | Polacrilex | 500 μm | 1.90% | 61.50% | 74.30% | 80.10% | 85.20% | 91.30% | 95.10% | 100.00% |
| | | spheres | | | | | | | | |
| 093021-4 | Polacrilex | 700 μm | 7.40% | 51.30% | 66.90% | 74.90% | 81.70% | 87.20% | 94.30% | 100.00% |
| | | spheres | | | | | | | | |
| 100621-1 | 12% | 200 μm | 2.70% | 73.60% | 84.80% | 87.70% | 90.60% | 93.80% | 97.70% | 100.00% |
| | Nicotine | spheres | | | | | | | | |
| | Solution | | | | | | | | | |
| 100621-2 | 12% | 350 μm | 5.10% | 80.20% | 87.30% | 91.30% | 93.50% | 95.60% | 98.20% | 100.00% |
| | Nicotine | spheres | | | | | | | | |
| | Solution | | | | | | | | | |
| 100621-3 | 12% | 500 μm | 4.50% | 74.60% | 85.70% | 90.80% | 93.50% | 96.00% | 98.50% | 100.00% |
| | Nicotine | spheres | | | | | | | | |
| | Solution | | | | | | | | | |
| 100621-4 | 12% | 700 μm | 3.60% | 70.40% | 79.10% | 85.30% | 89.50% | 92.80% | 97.40% | 100.00% |
| | Nicotine | spheres | | | | | | | | |
| | Solution | | | | | | | | | |
| 100621-5 | 12% | Particulate | 1.00% | 27.70% | 33.00% | 39.10% | 43.70% | 52.40% | 70.10% | 100.00% |
| | Nicotine | MCC | | | | | | | | |
| | Solution | | | | | | | | | |
| 100621-6 | Polacrilex | Particulate | 0.60% | 15.50% | 23.10% | 28.10% | 33.90% | 42.00% | 59.90% | 100.00% |
| | | MCC | | | | | | | | |

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A pouched product, comprising:
an outer water-permeable pouch defining a cavity; and a composition adapted for oral use situated within the cavity, the composition comprising at least one water-soluble component, a substantially spherical filler component in an amount of about 60% or more by dry weight of the composition, and water in an amount of about 30% or less by weight of the composition, wherein the average particle size of the substantially spherical filler component is 350 microns to 650 microns.

2. The pouched product of claim 1, wherein the average particle size of the substantially spherical filler component is 375 microns to 625 microns.

3. The pouched product of claim 1, wherein water is present in an amount of about 5% to about 30% by weight of the composition.

4. The pouched product of claim 3, wherein water is present in an amount of about 15% to about 26% by weight of the composition.

5. The pouched product of claim 1, further comprising at least one humectant in an amount of about 0.1% by weight or higher by weight of the composition.

6. The pouched product of claim 5, wherein the at least one humectant is present in an amount of about 0.5% by weight to about 15% by weight of the composition.

7. The pouched product of claim 5, wherein the at least one humectant is propylene glycol, glycerin, or a combination thereof, or wherein the at least one humectant is glycerin in the absence of propylene glycol.

8. The pouched product of claim 1, further comprising at least one sugar alcohol.

9. The pouched product of claim 8, wherein the at least one sugar alcohol comprises xylitol.

10. The pouched product of claim 9, wherein xylitol is present in an amount of about 0.1 to about 5% by weight of the composition.

11. The pouched product of claim 1, wherein the composition further comprises a sweetener, a salt, and a flavorant.

12. The pouched product of claim 1, wherein the substantially spherical filler component comprises microcrystalline cellulose.

13. The pouched product of claim 1, wherein the composition is substantially free of a tobacco material.

14. The pouched product of claim 1, wherein the composition comprises one or more active ingredients selected from the group consisting of a nicotinic component, nutraceuticals, botanicals, stimulants, amino acids, vitamins, cannabinoids, cannabamimetics, terpenes, pharmaceutical agents, and combinations thereof.

15. The pouched product of claim 1, wherein the composition comprises a nicotinic component selected from the group consisting of nicotine, a nicotine salt, a resin complex of nicotine, and combinations thereof.

16. The pouched product of claim 15, wherein the pouched product exhibits a nicotine dissolution rate characterized by at least 80% by weight of the nicotine within the product calculated as the free base eluting from the product within 20 minutes as determined by high performance liquid chromatography (HPLC) analysis.

17. The pouched product of claim 1, wherein the composition further comprises one or more components selected from the group consisting of one or more additional fillers, binders, pH adjusters, buffering agents, colorants, disintegration aids, antioxidants, and preservatives.

18. The pouched product of claim 1, wherein the spherical MCC comprises a coating layer comprising one or more components selected from the group consisting of a polyol, a functional cellulose, a filler, an active ingredient, a flavor, and combinations thereof.

19. The pouched product of claim 1, wherein the substantially spherical filler component comprises microcrystalline cellulose and has an average particle size of about 375 microns to about 625 microns, and the composition further comprises at least one humectant in an amount of about 0.5% by weight to about 15% by weight of the composition, xylitol in an amount of about 0.1 to about 5% by weight of the composition, and a nicotinic component in an amount of about 0.1% to about 10% by weight of the composition calculated as the free base.
